(19) United States
(12) Patent Application Publication
(10) Pub. No.: US 2014/0339161 A1
(43) Pub. Date: Nov. 20, 2014

(54) FLUID COMPONENT SEPARATION DEVICES, METHODS, AND SYSTEMS

(71) Applicant: The Trustees of Columbia University in the City of New York, New York, NY (US)

(72) Inventors: Edward Leonard, Bronxville, NY (US); Michael Hill, Wyckoff, NJ (US); Cees J.M. Van Rijn, Hengelo (NL); Ilan K. Reich, Short Hills, NJ (US); Levy Amar, Brooklyn, NY (US)

(73) Assignee: The Trustees of Columbia University in the City of New York, New York, NY (US)

(21) Appl. No.: 14/344,296

(22) PCT Filed: Oct. 8, 2012
(86) PCT No.: PCT/US12/59247

§ 371 Date: Mar. 11, 2014

Related U.S. Application Data
(60) Provisional application No. 61/544,913, filed on Oct. 7, 2011.
(60) Provisional application No. 61/635,370, filed on Apr. 19, 2012.

(51) Int. Cl. A61M 1/34

(52) U.S. Cl. CPC A61M 1/3403; A61M 1/34
USPC 210/637; 210/650

(57) ABSTRACT

A system for ultrafiltration employs a crossflow filtration module for extracting a fraction from a sample fluid (e.g., blood) and a recirculating permeate loop to produce a concurrent permeate flow through the filtration module to maintain a positive transmembrane pressure at all points of the crossflow filter. Permeate in the recirculating loop is enriched by a processing module and stabilized by removing an enriched fraction thereof. In an embodiment, the enriched fraction is concentrated plasma that is returned to a patient.

### CROSS REFERENCE TO RELATED APPLICATIONS

This application claims the benefit of U.S. Provisional Application No. 61/544,913, filed 7 Oct. 2011, and U.S. Provisional Application No. 61/635,370, filed 19 Apr. 2012, the disclosures of both of which are hereby incorporated by reference herein in their entireties.

### STATEMENT REGARDING FEDERALLY SPONSORED RESEARCH

This invention was made with U.S. government support under RO1 HL038306 awarded by the National Institutes of Health—National Heart, Lung, and Blood Institute; and under NIH 528801 awarded by the National Institutes of Health (NIH). The U.S. government has certain rights in the invention.

### BACKGROUND

Extracorporeal processing of blood is known to have many uses. Such processing may be used, for example, to provide treatment of a disease. Hemodialysis is the most commonly employed form of extracorporeal processing for this purpose. Additional uses for extracorporeal processing include extracting blood components useful in either treating others or in research. Apheresis of plasma (i.e., plasmapheresis) and thrombocytes, or platelets, are the procedures most commonly employed for this purpose. Also, non-therapeutic devices have been developed to analyze blood which may involve extraction of blood components. For example, some devices can separate blood and plasma, or specific analytes, for purposes of diagnosis.

Devices for separating and filtering components of all types of fluids are known. For example, filtration may be used to separate components for analysis or for production of food products. Microfluidic devices for separating and cleansing fluid components have been proposed. These types of devices pose technical challenges that are addressed by the presently disclosed subject matter.

### SUMMARY

Objects and advantages of embodiments of the disclosed subject matter will become apparent from the following description when considered in conjunction with the accompanying drawings.

### BRIEF DESCRIPTION OF THE DRAWINGS

Embodiments will hereinafter be described in detail below with reference to the accompanying drawings, wherein like reference numerals represent like elements. The accompanying drawings have not necessarily been drawn to scale. Where applicable, some features may not be illustrated to assist in the description of underlying features.

FIG. 1A is a top view of a microfluidic filtration device from a top view.

FIG. 1B shows the filtration device of FIG. 1A in side section view.

FIG. 1C shows a pressure profile characterizing an operational mode of the filtration device according to any of the embodiments of the disclosed subject matter.

FIG. 2 shows a crossflow filtration device with fluid lines, controller, and pumps.

FIG. 3 shows a mechanism for fluid separation using the filtration device of the disclosed embodiments in which a permeate flow is partly recirculated and partly extracted.

FIG. 4 shows the mechanism for fluid separation of FIG. 3 using the filtration device of the disclosed embodiments used to separate plasma from blood of a living patient.

FIG. 5 shows another mechanism for fluid separation using the filtration device of the disclosed embodiments in which a permeate flow is recirculated after further processing, in embodiments, producing a product flow.

FIG. 6 shows another mechanism for fluid separation using the filtration device of the disclosed embodiments in which plasma is separated from blood and the plasma is ultrafiltered or otherwise processed, optionally, depending on the processor, producing a waste stream and a flow of permeate is combined with the retentate outflow to balance a change in properties of the permeate stream.

FIG. 7 shows another mechanism for fluid separation using the filtration device of the disclosed embodiments in which a fraction of a recirculating permeate is returned to the retentate flow in a concurrent flow arrangement.

FIG. 8A shows another mechanism for fluid separation using the filtration device of the disclosed embodiments in which a fraction of a recirculating permeate is returned to the retentate flow in a concurrent flow arrangement.

FIG. 8B shows an alternate embodiment based on that of FIG. 8A in which flow directions are reversed so that the plasma separation and return are switched between modules.

FIG. 9 shows another mechanism for fluid separation using the filtration device of the disclosed embodiments in which a fraction of a recirculating permeate is returned to the retentate flow in a counterflow arrangement.

FIGS. 10A through 10C illustrate aspects of cross-flow filtration according to the prior art.

FIG. 11A and 11B illustrate aspects of an embodiment in which plasma extraction and return are performed in a single module.

FIGS. 12A and 12B illustrate features of a blood component separation device in which concurrent flow is in a recirculating channel flows across flow restrictions between a side wall and vertical channel walls so that the static pressure drops in stepwise fashion for each of a number of successive portions of a crossflow filter.

FIG. 12C illustrates a pressure profile of the embodiments of FIGS. 12A and 12B.

FIGS. 13A and 13B illustrate a fluid component separation module mechanical features.

FIGS. 14A and 14B illustrate features for periodic return of fluid components to maintain balance of the components in an extraction stream.

FIGS. 15A, 15B, and 15C illustrate features for compensating for changes in mass of permeate and retentate stream flows.

FIG. 16 illustrates a schematic of an ultrafiltration system.

FIG. 17 shows a method for configuring a crossflow filter for use in the application of FIG. 16 and other embodiments disclosed herein.

FIG. 18 shows experimental data representing a critical TMP.

FIG. 19 shows an example of an ultrafiltration system with quantitative parameters superimposed on it.

FIGS. 20A, 20B, and 20C show alternative methods of returning blood to a patient that may be used to vary embodiments disclosed herein.

FIGS. 21A and 21B show alternative access devices permitting permeate to be returned to a patient.

FIG. 22 shows a variation of the embodiment of FIG. 16 and other embodiments disclosed herein in which the permeate processor includes, or is, a dialyzer.

FIG. 23 shows a variation of a filter design that may be used to support or fully integrate the channeling of permeate for a separation device.

FIGS. 24 and 25 illustrate further variations of the filter embodiments discussed with reference to FIG. 23.

### DETAILED DESCRIPTION OF THE DRAWINGS

Effective microfluidic cross-flow filtration may be limited by streamwise pressure loss in the retentate channel. In microfluidic channels, in the presence of high shear rates, the pressure difference across the cross-flow filter can be much higher at the upstream end of the cross-flow filter than at the downstream end. If the upstream pressure is adjusted to make it low, there may result a backflow of filtrate at the downstream end of the filter. In cross-flow filtration of fluids that have very high particle fraction, such as whole blood, high upstream pressure may lead to compaction of filtered material on the filter surface. For example, particle fraction of blood is the volume fraction of cytoplasmic bodies in the whole blood. The compaction results in blockage of the open area of the filter. In many configurations and operational regimes, including multi-phase fluid properties, there may not be a window of operability between these extremes.

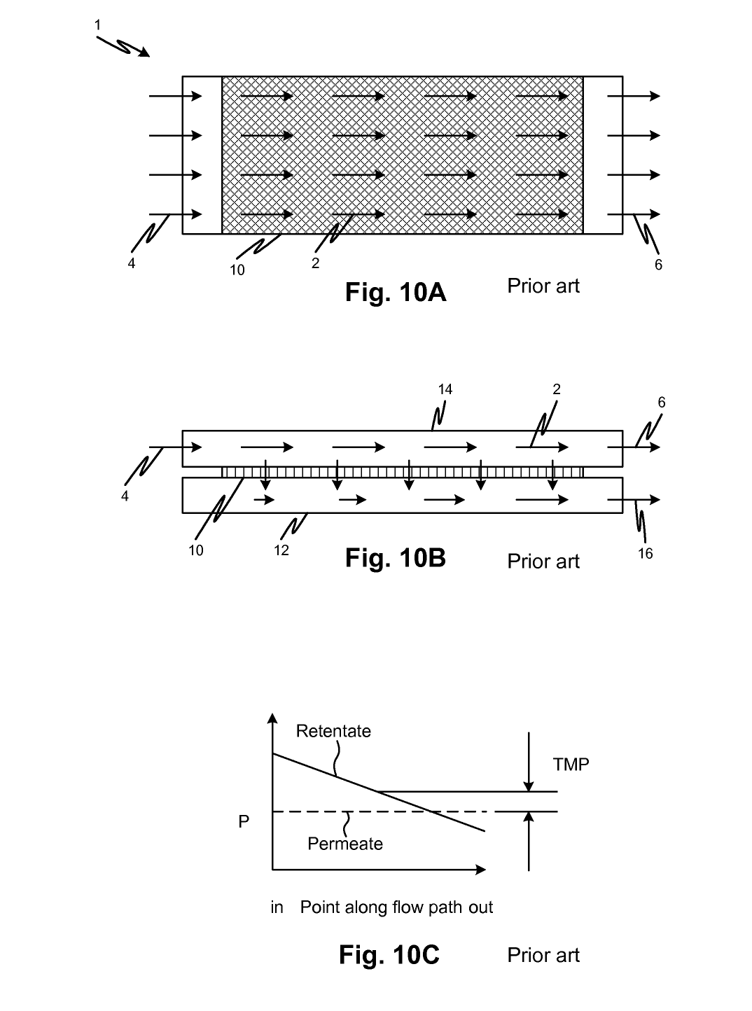

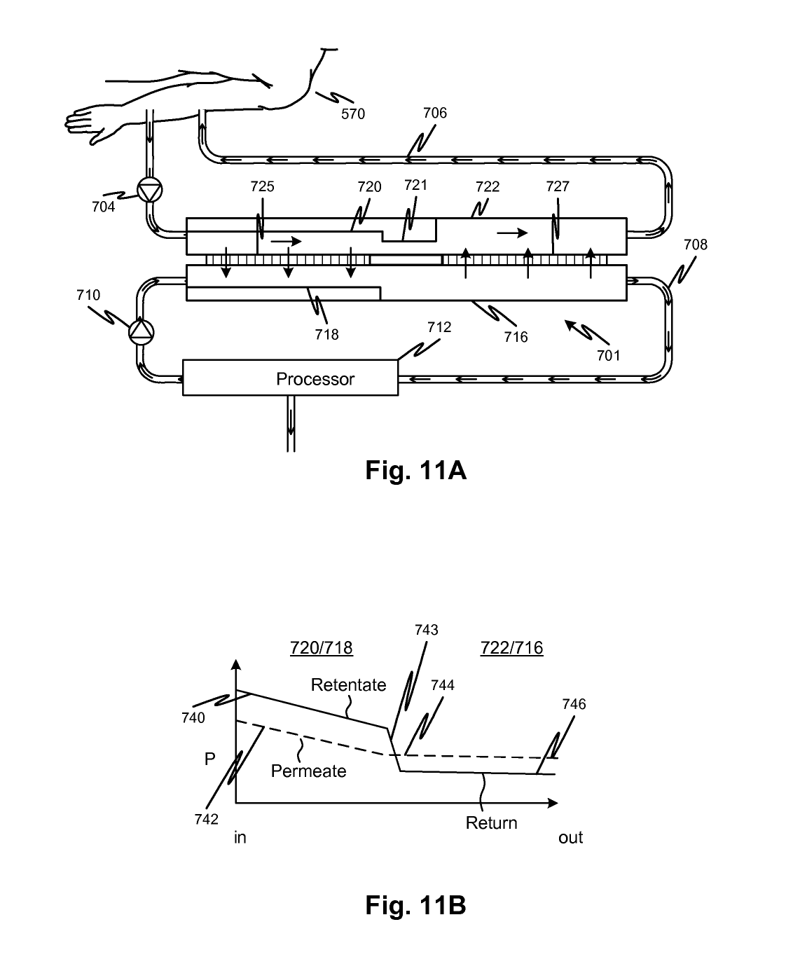

FIGS. 10A through 10C illustrate aspects of cross-flow filtration according to the prior art. In prior art cross-flow filtration systems, the particle volume fraction is generally of very low magnitude (1 percent or less). A cross-flow filtration channel 1 has a filter 10 across which a flow of particle-bearing fluid 2 occurs. The material enters the channel 14 as indicated at 4 and leaves as indicated at 6 such that there is a continuous sweeping of the filter 10 surface, which keeps particles from accumulating on it. Permeate passes through the filter 10 into channel 12, and leaves it, as indicated at 16. As a result of this configuration, there may be significant loss of pressure in the streamwise direction so that the transmembrane pressure (TMP) is initially high and drops progressively along the channel as illustrated in FIG. 11C.

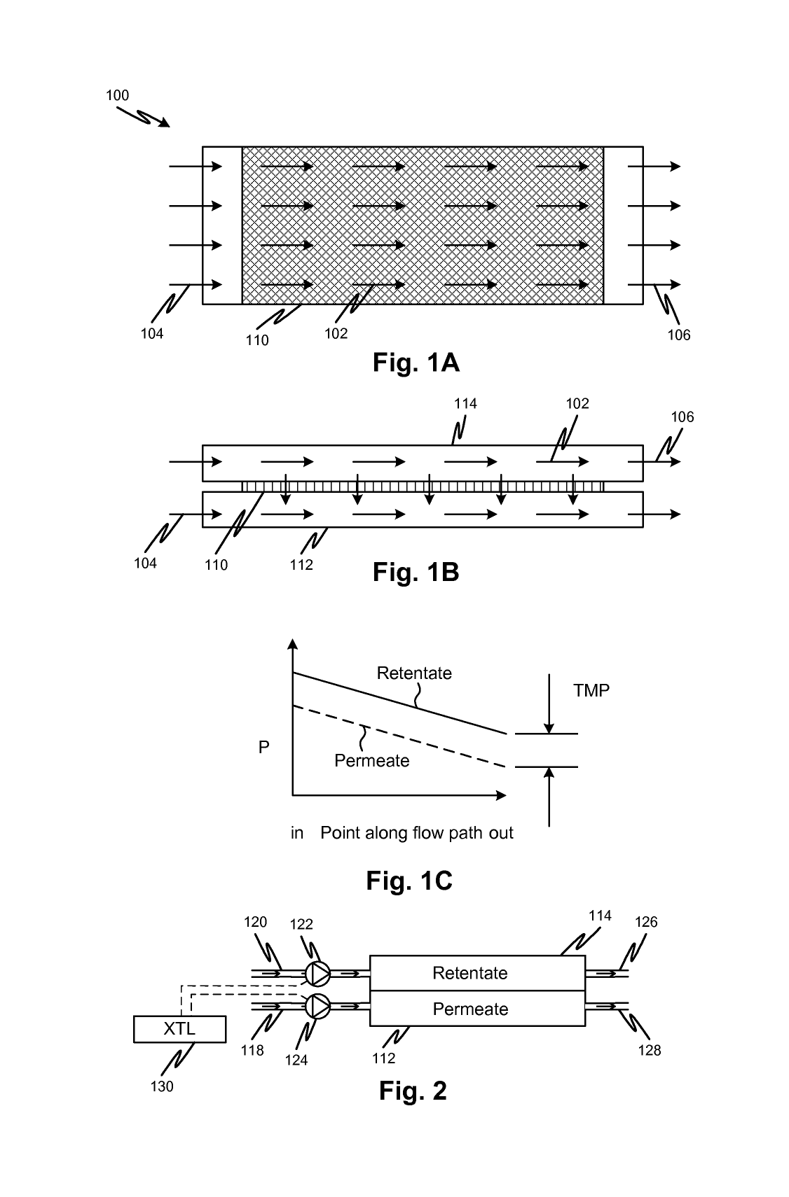

In a high particle volume fraction fluid, it has been determined by experiment, that exceeding a certain critical TMP at the upstream end of a microfluidic retentate channel can cause an entire filter to clog up due to a runaway instability effect in which as the excess TMP clogs the upstream end, the high TMP region moves downstream, clogging the remaining part of the filter and so on until no filtration occurs at all. It has been discovered that this effect can be mitigated by flowing the permeate in a concurrent flow relationship with the flow in the retentate channel as now discussed with reference to FIGS. 1A through 1C.

Referring now to FIGS. 1A through 1C, a filtration device 100, a retentate channel 114 is bounded in part by a filter 110, preferably a micro-sieve type filter suitable for cross flow filtration and having uniform pore size with non-branching straight channels and presenting a polished smooth surface interrupted only by precisely machined openings, for example of 0.5 to 1.0 micron diameter. Such micro-sieve devices are known and frequently used as polishing filters in the food industry. A flow of fluid to be filtered enters as indicated at 104 and leaves at 106. In the cross-flow, indicated at 102, a shear force sweeps particles from the filter surface. As shown in FIG. 1B, a continuous flow in a permeate channel 112 produces a high shear rate that can produce a streamwise pressure drop through the channel that produces a constant TMP along the streamwise length of the filter 110 despite the streamwise pressure drop. This is because the permeate pressure drop in channel 112 can have a magnitude that is substantially the same as that in the retentate channel 114. The continuous flow in the permeate channel 112 is provided by an inlet flow indicated at 104. The pressure profile in the retentate and permeate channels are illustrated in FIG. 1C.

FIG. 2 shows a configuration in which respective pumps 122 and 124 for the cross-flow fluid and the permeate channel fluid, respectively, provide a flow rate such that, for the filtering device configuration, results in a nearly constant TMP along the streamwise axis. Outlet channels 126 and 128 convey fluid from the filtration device. A controller 130 controls pumps 122 and 124 to ensure flow conditions that provide for the generally constant TMP. Note that instead of a constant TMP, the range of TMPs may fall within a discrete range and in further embodiments, in a discrete range that varies by no more than 50% and is positive at all points along the extend of the filter.

In embodiments, the crossflow filtration configuration of FIG. 1B or FIG. 2 (as well other embodiments to be described below) flows human blood on the retentate side and plasma is flowing on the filtrate side. The rate of flow of blood may be chosen to be the highest rate that, for the channel height, does not cause hemolysis. In alternative embodiments, the rate may be substantially lower than that rate to ensure a margin of safety. In embodiments, the channel height is between 500 microns and 100 microns and the width of the channel is several times the height. The height and width are the axes that are normal to the direction of flow. Further the transmembrane pressure (TMP) may be chosen to prevent cells from sticking to the retentate side of the filter such that the shear created by the flow continuously sweeps cells along the filter surface. In embodiments, the TMP may be substantially uniform along the length of the filter. In further embodiments, the TMP is at no point less than zero (flow is at all points on the surface of the filter in one direction). Appropriate coatings may be employed to improve the effect of shear sweeping the cells across the surface.

In further embodiments, the volume fraction of particles in the retentate flow is more than 10% and in further embodiments it is the fraction in whole blood. In variations of the above, blood is prediluted to reduce the volume fraction, using blood normal solution or plasma. In embodiments, the flux rate across the filter is between 5 and 20 percent of the flow rate of the retentate flow. In embodiments, blood from a living patient flows continuously in the channel 114 for days without substantial destruction of cells in the blood. In further embodiments, the plasma is ultrafiltered to 5 to 20 percent of the volume by removal of water and solutes and the reduced product returned to the living patient or, alternatively, the plasma is recirculated at a volume rate that is higher or lower than the rate of blood flow in order to generate the permeate flow pressure profile shown in FIG. 1C.

By providing a flow through the permeate channel, it is possible to mitigate the above-described undesirable effects by reducing the variation in the transmembrane pressure along the streamwise axis of the cross-flow filter. Pressure drop along the retentate channel suspension channel is offset by a pressure drop along the permeate channel in which a concurrent flow is established.

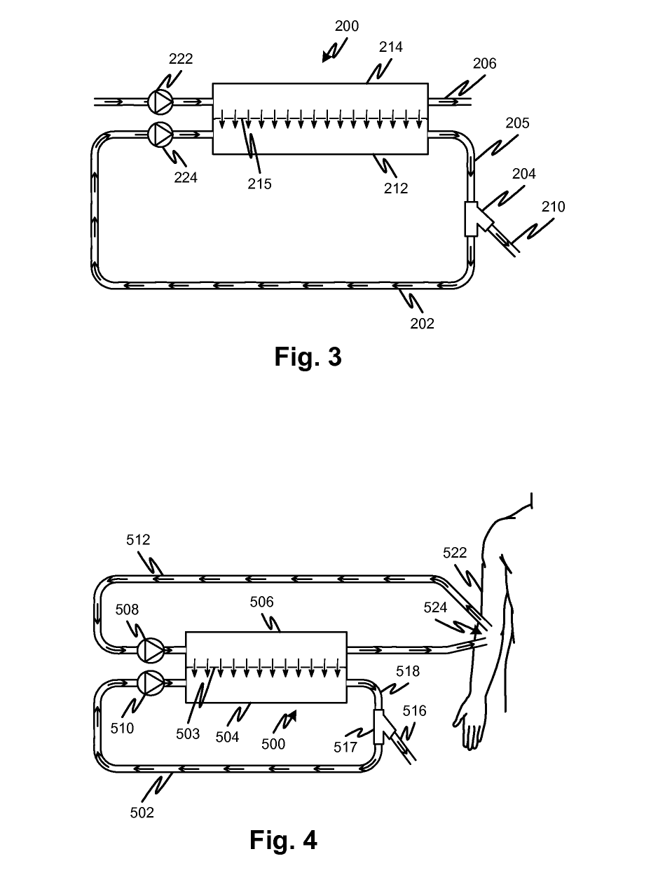

Referring to FIG. 3, a mechanism for fluid separation uses a filtration device 200 with concurrent flowing permeate and retentate channels 212 and 214. A fluid containing a particulate flows in by means of a pump 222 (which may be controlled by a controller) to flow into and out (through line 206) of the retentate channel 214. A crossflow filter 215 separates the retentate channel 214 from the permeate channel 212. The flow in the permeate channel 212 is controlled by pump 224. The permeate and retentate flow rates are regulated such that a restricted range of TMP is generated across the filter 215. In this and any of the other embodiments, the filter may be a micro-sieve type filter suitable for cross flow filtration and having uniform pore size with non-branching straight channels therethrough. The exiting permeate 205 is circulated in a return channel 202 to generate the flow and pressure drop in the permeate channel that provides the restricted range of TMPs (e.g., substantially constant TMP as illustrated in FIG. 2). A fraction of the recirculating flow may be extracted by a junction 204 to form a product stream 210. In embodiments, the system of FIG. 3 is used for filtering plasma from whole blood such that the product stream 210 is plasma as discussed with reference to FIG. 4.

FIG. 4 shows the mechanism for fluid separation of FIG. 3 using the filtration device 500 in an application for separating plasma from the blood 512 of a living patient 522. In this embodiment, a permeate flow is partly recirculated, partly extracted, and partly returned to a retentate flow. Whole blood is extracted by a blood pump 508 and conveyed to the retentate channel 506 where it is subject to cross-flow filtration by a filter 503 and returned to the patient. A fraction of the plasma from the permeate flow is drawn through the cross-flow filter 503 to form a permeate flow 518 in the permeate channel 504 using a plasma pump 510. In the present and the other embodiments in which blood is filtered, the cross-flow filter may have pores that are sized to block the passage of cytoplasmic bodies while permitting macromolecules to pass. For example the openings of a microsieve chip may be in the range of 0.1 to 2 microns and in the range of about 0.2 to 1 micron. Most of the permeate is recirculated continuously in a channel 502 by the pump 510. The recirculating flow generates a streamwise pressure loss in the permeate channel 504. A product fraction 516 of the recirculating permeate flow may be drawn from a junction 517.

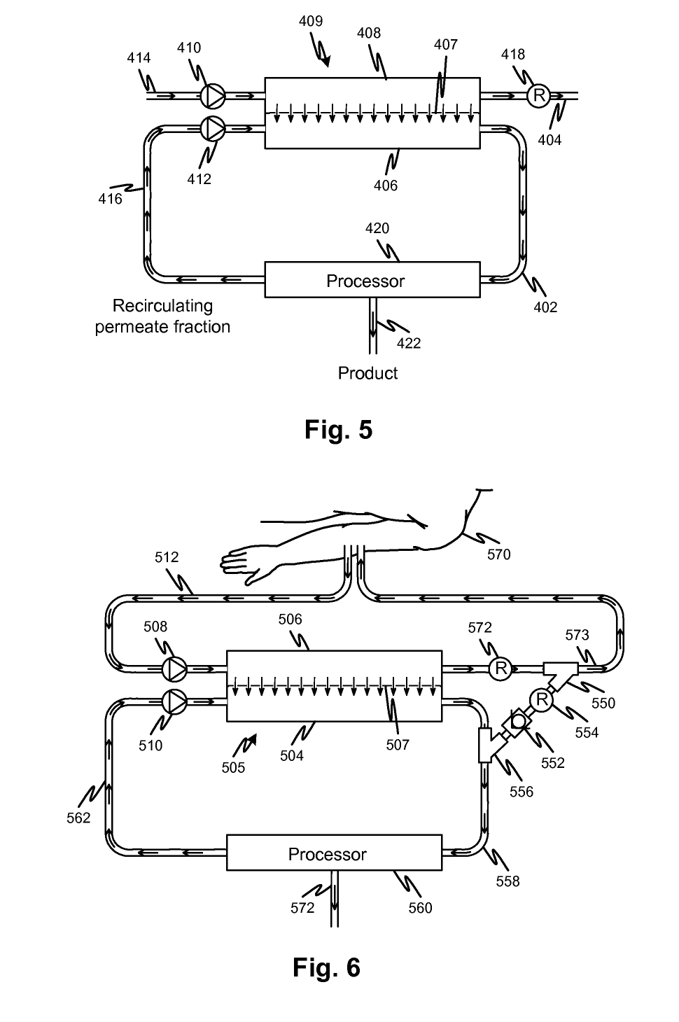

Referring now to FIG. 5, a filtration device 409 has retentate 408 and permeate 406 channels. The retentate channel 408 receives a fluid from a source 414 via a pump 410 into a retentate channel 408 of component separation module 409. A permeate fraction flows through a filter 407 into the permeate channel 406. A flow restrictor 418 may generate resistance to aid in the generation of a selected TMP depending on the configuration of the flow system. The permeate flow in recirculation channel 402 recirculates back to the permeate channel 406 driven by pump 412 to produce a pressure loss in the permeate channel 406. This provides the ability to mitigate the change in TMP along the streamwise axis of the filter 407. The permeate flow 402 is conveyed to a processor 420 that alters the properties of the permeate flow resulting in a modified permeate stream 416 which is recirculated. The processor may generate a product stream 422. For example, the processor may be a filter with a smaller pore size than the filter 407 such that the product stream 422 is a filtrate of the permeate stream. As in the embodiment of FIG. 6, the primary flow 414 may be whole blood from a living patient and the product stream 422 may be water and uremic waste with the overall function being ultrafiltration of a living patient.

A problem that may arise in the configuration of FIG. 5 is that during a long operational cycle, material in the permeate channel 402 may change composition affecting the performance of either the processor 420, the filtering device 409, or the product stream 422. In certain applications, this effect may be balanced as shown in the embodiment of FIG. 6. In the embodiment of FIG. 6, the processed fluid is blood, but in other embodiments it may be any fluid continuing a suspension of particulates. Referring now to FIG. 6, blood is extracted from a patient 570 and flows in an arterial channel 512 in the manner common to extracorporeal blood treatment. The whole blood flows to the retentate channel 506 of the component separation device 505 pumped by pump 508. The latter may be a peristaltic pump and pressure pulse isolator or damper. Plasma flows across the filter 507. The permeate (plasma) recirculates in channel 558 through processor 560 and is returned, urged by pump 510, to the permeate channel 504. In the present example, processor 560 may be, or include, an ultrafilter to remove water and uremic toxins from the plasma.

In the case of an ultrafilter being used as the processor 560, the flow of plasma, including macromolecules, across the filter 507 is balanced by a stream of filtrate 572, but the macromolecules are retained by the ultrafilter. This causes the macromolecules to concentrate in the recirculating plasma (permeate) flow. To balance what would otherwise be continuously concentrating material retentate in channel 558, for long term treatment embodiments, a fraction of the concentrated plasma flows through a branch 556 back to the return blood stream 573 via a junction 550. In an alternative embodiment, the return plasma flows back to the arterial line 512 for immediate flow through the separation module 505. A flow restrictor 554 may be provided along with a check valve 552. A further flow restrictor 572 may also be provided to create a flow resistance in the retentate stream exiting the filtration device 505. By appropriately controlling the pumps 508 and 510 and assuming appropriate selection of the flow restrictors 554 and 572, a net flow of macromolecules in a concentrated flow can be returned to the blood of the patient while still permitting a net flow of water and crystolloid solutes to be extracted by the processor 560 for an indefinite period.

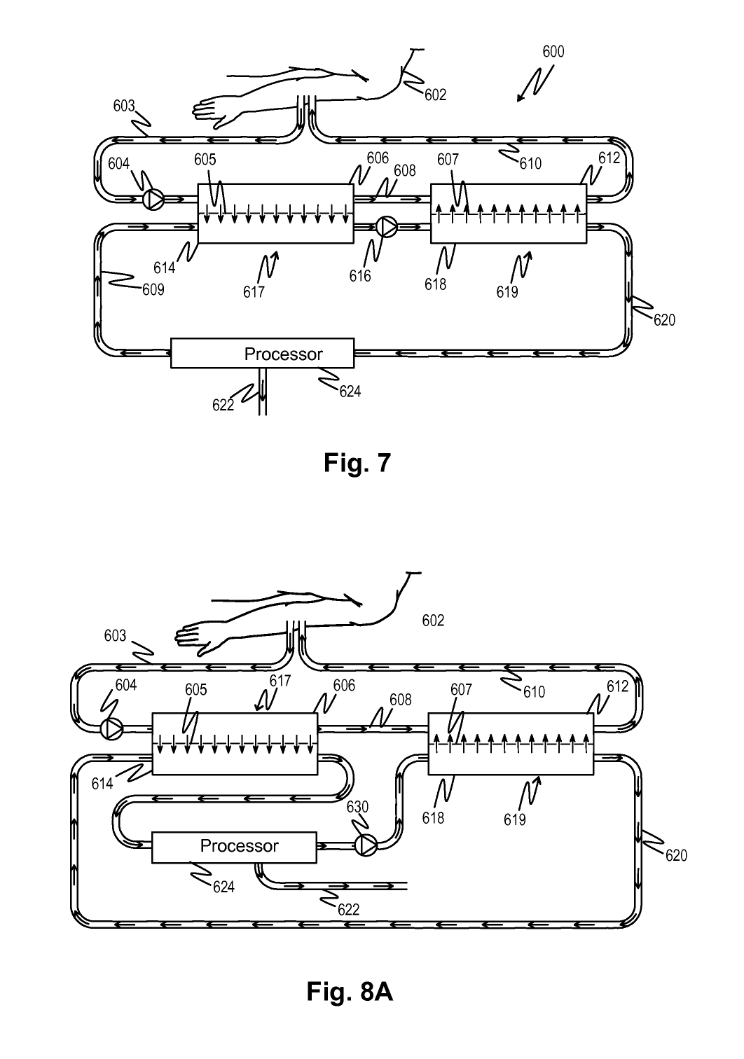

Referring now to FIG. 7, in system embodiment 600, blood 603 is extracted from a patient 602 and pumped by pump 604 to a retentate channel 606 of the component separation module 617. Plasma flows across a filter 605 in the module 617 that separates the retentate channel from a permeate channel 614. The permeate (in the example case, plasma) recirculates in channel 620 through processor 624 and is returned, urged by pump 616, to the permeate channel 614. In the present example, processor 624 may include an ultrafilter. The flow of plasma, including macromolecules, across the filter 605 is balanced by a stream of filtrate 622, but the macromolecules are retained by the ultrafilter of processor 624. A fraction of the permeate flow in line 620 amount of plasma flows back to the retentate stream 608 through a further filtration device 619. This returns the plasma, concentrated in high molecular weight (HMW) components that do not pass through the membrane of the ultrafilter in processor 624, back to the patient. Since the concentration of HMW components is higher in the returned stream, the flow rate of the return stream need only be a fraction of the primary permeate flow rate through the filter 605 in order maintain an equilibrium concentration of HMW components in the plasma stream 620.

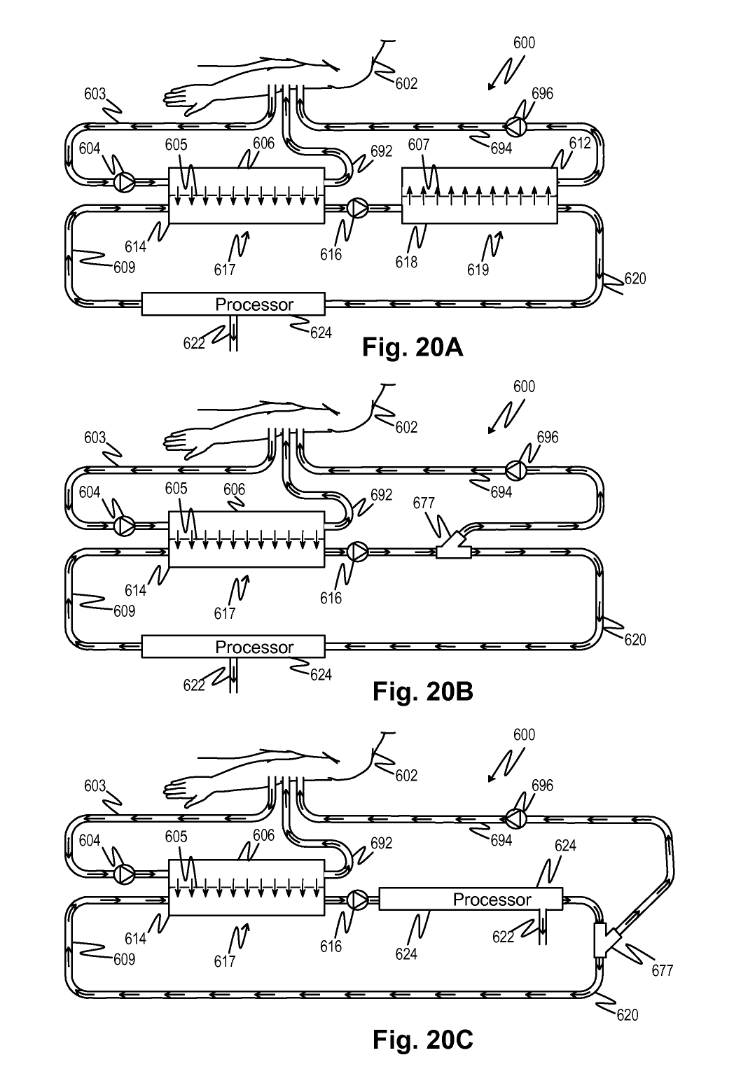

To return the concentrated HMW component stream, the pump 616 creates a positive TMP across filter 607 thereby flowing plasma, concentrated in macromolecules, from a filtrand channel 618 to a filtrate channel 612 and on to a venous line 610 for return to the venous line 610 and on to the patient 602. Flow restrictors may be provided as required to achieve the required driving pressures. The separation module 619 may function in the same manner as separation module 614 in having a cross-flow configuration with a limited range of TMP. In an alternative embodiment, the separation module 619 is replaced by a simple chamber with a microsieve filter on the lower side 618 where the sweeping effect used to clear cytoplasmic bodies from the filter 605 of separation module 617 is not needed. The filtrate side 612 however may benefit from such sweeping effect but it may not be necessary, depending on the particular conditions. Thus, filtrand and filtrate sides 618, 612 may be flow plenums without substantial pressure drop, in embodiments. In alternative arrangements, the return flow of concentrated plasma is pumped directly to a second venous line to the patient while blood returns through a first venous line, rather than forming a mixed stream. This embodiment is illustrated in FIG. 20A, where a blood return line 692 flows blood to the patient and a plasma return line 694 flows concentrated plasma back to the patient 602. A pump 696 may be used to control the rate of plasma flow but may not be necessary if the TMP in module 619 can drive the flow. This variation may be applied to any of the applicable embodiments described herein and is illustrated here using FIG. 7 as an example only. As shown in FIG. 20B, the return flow can also be passed using a simple junction as indicated at 677. The junction 677 may incorporate a check valve for the return flow in line 694. In embodiment 20C, a processor 624 is positioned prior to the junction 677. The variations of the junction 677 and the processor 624 as described herein apply to this embodiment as well, for example, the processor may include a ultrafilter, a dialyzer with a dialysis flow (in which case inlet as well as outlet lines 622 would be provided), adsorbents, etc. By positioning the processor 624 prior to the junction 677, the property of the permeate flow that is enhanced by the processor (depending on the processor configuration) will be more enriched in the return flow 694 than in the embodiment of FIG. 20B. This may allow the balance of the property, for example the concentration of HMW species in the permeate line, to be lower.

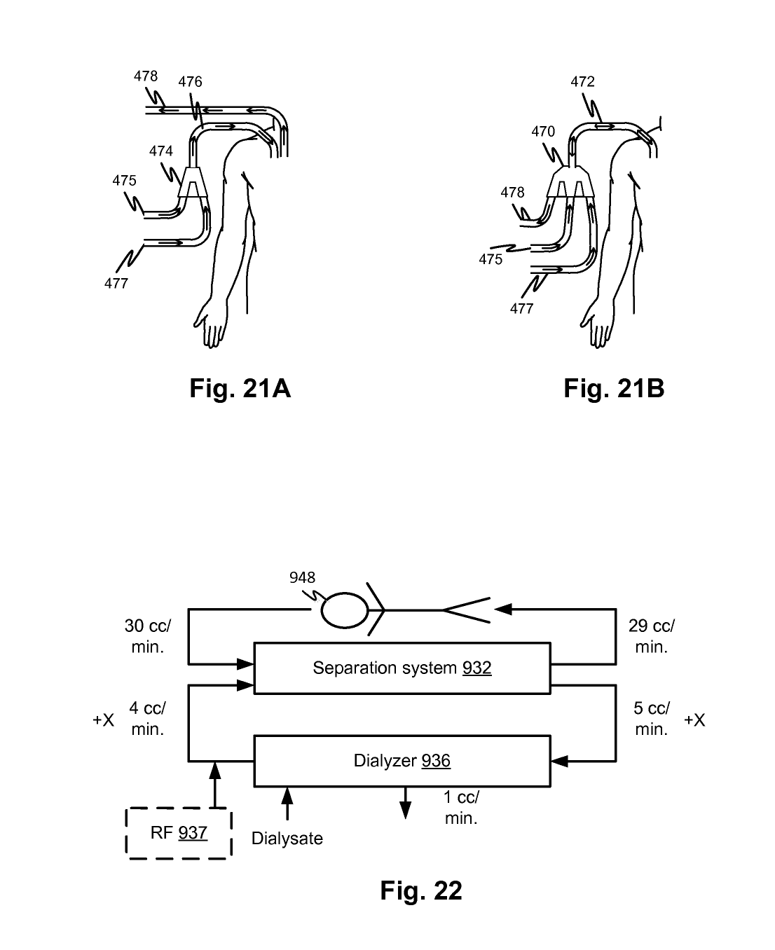

As in the embodiments of FIGS. 20A and 20B, the blood access and returns can be direct to the patient in any of the disclosed embodiments, where applicable. The patient access may be a single triple lumen catheter or cannula. Alternatively, in a “two-port” access, the return (venous) flow may be provided by a dual lumen catheter or cannula and the outgoing (arterial) flow may be by a single lumen catheter or cannula. In another variation, three cannulae or catheters are employed. In yet another variation, the return (venous) flow is accomplished using a single lumen of a dual or single lumen device with a converging junction at the access. FIG. 21A shows a single lumen functioning as an arterial line 478 connected to a patient access and a junction 474 joining permeate 475 and blood 477 return lines leading to a patient access (a central line being illustrated). FIG. 21B shows a triple junction 470 which may join return permeate 475 and blood 477 return lines to one lumen of a dual lumen line 472 (or 472 may be a single lumen for intermittent flow) and a outgoing arterial line 478.

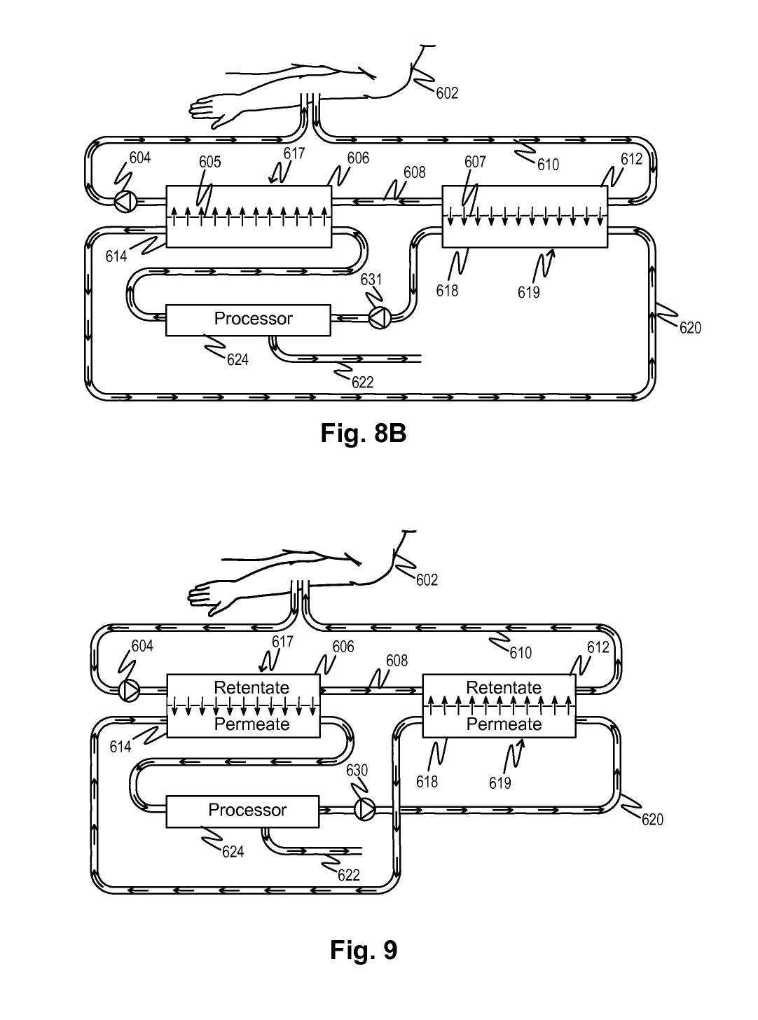

Referring now to FIG. 8A, the configuration is similar to that of FIG. 7. Blood 603 is extracted from a patient 602 and flow to the retentate channel 606 of the separation module 617. Plasma flows across the filter 605. The permeate (plasma) recirculates in channel 620 through processor 624 and is returned, urged by pump 630, to the permeate channel 614. The pump 630 creates a positive TMP across filter 607 thereby flowing plasma, concentrated in HMW components, from a filtrand channel 618 to a filtrate channel 612 and on to a venous line 610 for return to the patient 602. Flow restrictors may be provided as required to achieve a balanced steady state condition. The processor 624 is positioned between the separation module 617 and the separation module 619.

As in the prior embodiment, in an alternative embodiment, the separation module 618 is replaced by a simple chamber with a microsieve filter on the lower side 618 where the sweeping effect used to clear cytoplasmic bodies from the filter 605 of separation module 617 is not needed. The filtrate side 612 however may benefit from such sweeping effect but it may not be necessary, depending on the particular conditions. Thus, filtrand and filtrate sides may be simple flow chambers, in embodiments.

Referring to FIG. 8B, in another embodiment, structurally similar to that of FIG. 8A, the flow of plasma is reversed so that module 619 functions as a component separation module in which plasma permeates the filter 607 and module 614 functions to return concentrated plasma to the venous blood flow through the filter 605. Correspondingly the flow of blood is also reversed and in this embodiment, the pump 604 may be reversible. The reversal may be done periodically. A benefit of the reversal is that the flow through filters 605 and 607 is periodically reversed which may help to remove any deposits due to filtration. In this embodiment, the pump 631 may be reversible. In an alternative embodiment, the flow of plasma and blood may be reversed by using flow reversing valves instead of reversible pumps. Referring to FIG. 9, the arrangement is essentially the same as that of FIG. 8A, including the identified variants, except that the flow of permeate 620 flows countercurrently through the separation module 619.

In the embodiments of FIGS. 6 through 9, flow resistance may be provided in the blood flow paths downstream of the retentate channel of the filtration device so that blood passes through it before returning to the donor. An additional flow resistance may be provided in the plasma flow path downstream of both the permeate channel of the filtration device and in the channel that recirculates plasma, so that at least some of the plasma from the plasma channel flows directly to processor where the processor is a filtration device such as an ultrafilter. The concentrated plasma from the plasma channel may also pass through an additional flow resistance before being returned to the animal or person. Such resistance may be provided by the second filtration devices of FIGS. 7 through 9. The magnitude of additional flow resistance in the plasma flow path may be such that the pressure drop across this resistance is approximately equal to sum of the required discharge pressure, the desired TMP in the dialyzer, and one half the pressure drop down the dialyzer. The magnitude of the additional flow resistance in the blood flow path may be such that the pressure drop across this resistance is approximately equal to sum of the desired TMP on the filter between the blood channel and plasma channel, and the pressure drop across the additional flow resistance in the plasma flow path.

An alternative to recirculating the permeate to provide a large concurrent flow to produce a pressure drop in the permeate channel is to shape the permeate channel such that sufficient streamwise pressure drop in the permeate channel occurs due to the permeate flow alone. An alternative to recirculating the permeate to provide a large concurrent flow to produce a pressure drop in the permeate channel is to shape the permeate channel such that sufficient streamwise pressure drop in the permeate channel occurs due to the permeate flow alone. For example, in a permeate channel tapered from a narrow height at the inlet end to a taller height at the outlet end, an initially small permeate flow in the streamwise direction is attended by a concomitantly high resistance and resulting streamwise pressure drop. If the retentate flow is being maintained at a constant level, also the pressure drop along the retentate channel has a constant value. This same pressure drop along the permeate channel can be maintained (herewith enabling a constant TMP along the filter) provided that the permeation rate is constant and the tapering of the permeate channel is properly adapted to enable this.

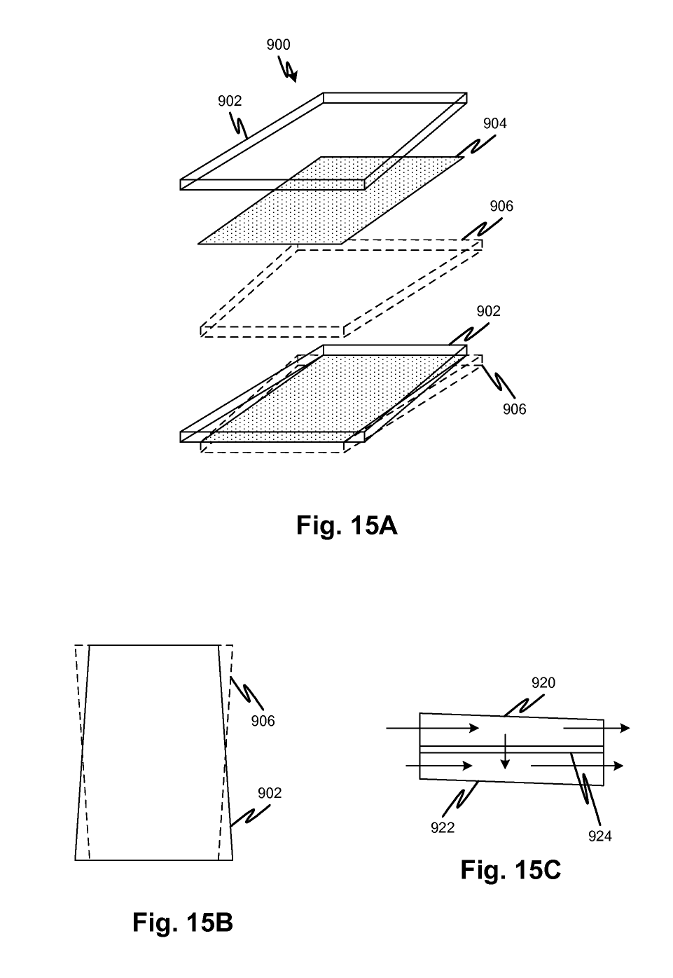

In embodiments, the passive concurrent flow configuration may be achieved within the body of a micro-sieve chip. The flow resistance scales cubic with the permeate channel height, thus tapering the channel height is preferred. For example the bottom of the permeate channel can be designed to have sufficient tapering to enable passive concurrent flow. Since there is a loss of flow from the retentate channel, the retentate channel may also be tapered so that its cross-section diminishes in a streamwise direction. Some other variations are illustrated in FIGS. 15A, 15B, and 15C. A separation module 900 has a retentate channel 902 that is tapered in plan view such that the flow area progressively drops in the streamwise direction. The retentate channel 902 is separated from a permeate channel 906 by a filter 904. The permeate channel progressively expands in the streamwise direction such that the flow area progressively increases from inlet to outlet; the flow resistance scales here are only (inversely) linear with the permeate channel width. In another preferred variation, a retentate channel's 920 depth decreases while a permeate channel's 922 depth increases. In this way the flow area of the retentate channel 920 increases progressively while the flow area of the retentate channel increases. Additional embodiments may be created by varying both the depth and the width of either or both channels to achieve the progressive flow area change described. The rate of change of flow area for either of these embodiments may be designed such that the TMP is closer to linear for both channels. The configuration may also be used to achieve other TMP profiles (TMP versus displacement in the streamwise direction).

For plasma filtration embodiments of the concurrent flow embodiments a flow rate in the retentate channel (e.g., 606 in FIG. 9) the flow rate may be 10 ml/min. The rate of plasma extraction may be 15 percent of that volume or 1.5 ml/min. The rate of recirculating co-flowing permeate may be selected based on the channel configuration and may be, for example 50% to twice the rate in the retentate channel. The ultrafiltration rate may be approximately 50 to 100 percent of the permeate flow rate through the cross-flow filter. It has been confirmed by experiment that cross-flow filter permeate flow rates through the cross-flow filter of 0.3 and up to 1.0 ml/min-cm² may be provided with stable operation using citrated whole blood.

In an initial startup of a system, a substitute fluid may be used in the recirculating permeate channel until the volume of permeate builds up and displaces it. For example, in a blood treatment system, a blood-normal fluid such as sterile dialysate may be used to prime the blood and plasma channels before the introduction of blood. The flow of permeate may displace the priming fluid as well. Alternatively, or in addition, plasma from an outside source may be used to prime the recirculating permeate channel.

The elements of the disclosed fluid circuits may be combined to form modules of a simplified system. That is, although depicted as interconnected filtration devices, it is possible to combine the filtration devices 617 and 619 of FIG. 9, for example, into a single unit with similar flow dynamics. An example of a module 701 of this configuration is illustrated in FIG. 11A. Referring now to FIGS. 11A and 11B, a pump 704 conveys fluid from a source, such as whole blood from a living patient 570, to a retentate channel 720 that transitions through a neck region 721 to a plasma return filtrate channel 722. The neck region 721 causes a pressure change thereacross due to the narrow size of the flow area. The filtrate channel 722 has a larger flow area than the retentate channel 720. The permeate channel 718 has a return flow through channel 708 which may subject the flow to processing by processor 712 as described any of the foregoing embodiments. The filtrand channel 716 may have a larger cross-section since no streamwise pressure change is required for return of concentrated permeate to the primary channel 706. A pump 710 moves fluid through the permeate channel 708. FIG. 11B shows a pressure profile through the module 701. In channels 720 and 718, retentate and permeate flow with progressive pressure loss as indicated by curve portions 740 and 742. A rapid loss of pressure occurs in the permeate flow as indicated at 743 due to the narrow size of the neck region 721. This drop in pressure causes flow across the filters 725 and 727 to be in opposite directions so that the permeate flow is now at a higher pressure than the retentate flow as indicated by curve portions 746 and 744 so that the permeate then flows across the filter 727 back into the channel 722.

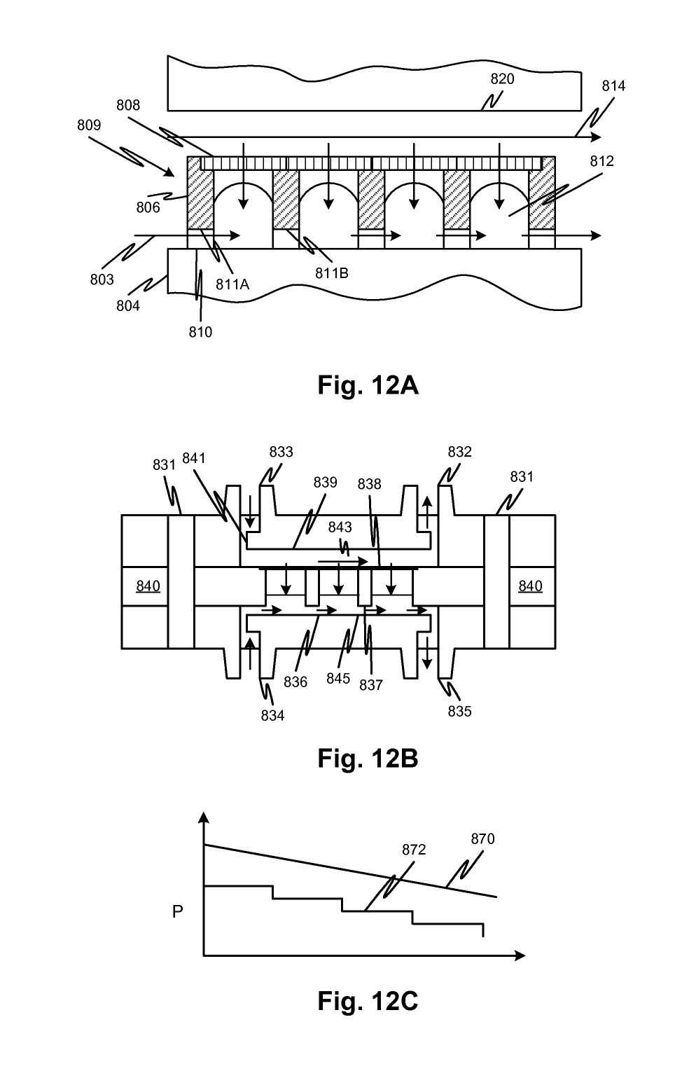

FIGS. 12A and 12B illustrate a blood component separation device in which a filter portion 809 has a structure that presents a polished and smooth surface 808 to blood to help ensure that cells do not stick to the filter. In addition, it is desirable, in some embodiments, for the depth of the filter to be minimal to permit free flow of plasma. Further, it is also desirable for the filter to be strong enough to withstand a TMP of 20-50 Torr to provide efficient use of the filter area, since the filters themselves can be expensive components. Still further, it is desirable for the filter to be stiff so that precise and repeatable pressure loss profiles can be achieved along the streamwise extent of the filter, thereby to achieve an optimal TMP over the entire filter. In the embodiment of FIG. 12A, structural members 806, provide precise flow areas that create flow bottlenecks, two of which are indicate at 811A and 811B for the recirculating flow of permeate 803. The precise flow area allows a positive TMP to be generated between the retentate flow 814 in retentate channel 820 and the permeate flow 803 along the streamwise length of the separation module. The recirculating permeate fluid may flow through the bottlenecks 811A and into plenum areas 812 to mix with incoming permeate flow. In alternative embodiments, the support structure of the filter is spaced apart from the wall 810 of the permeate channel such that flow bottlenecks are formed between the wall 810 and structural members 806 of the support structure of the filter. The effect is that the pressure profile of the retentate channel provides a uniform shear force to ensure that retained content is continually swept by the flow of the primary stream and recirculating permeate follows a stepwise curve. In the permeate channel this stepwise pressure drop may not be important, for example, in the blood ultrafiltration application, because it is not necessary to provide a surface shear on the permeate side. The pressure profile is illustrated in FIG. 12C. The permeate pressure profile is indicated at 872 and the retentate profile is indicated at 870. A characteristic of the embodiment of FIG. 12A is that the structural members that support the filter create the precise pressure loss profile in the permeate channel. This may allow lower recirculating flow rates in the permeate flow. The filter portion 809 may be integral with the structural members 806. The pores of the filter portion 809 may have axial lengths that are no more than 2 times their diameter. This makes the filter portion 809 very thin for 0.6 micron pores but the structural members may provide support. The structural members may be, for example, 500 microns thick (dimension along and transverse to the flow). The area between them may contain 300-1000 pores.

FIG. 12B shows a separation module based on the embodiments described with reference to FIG. 12A. The primary flow enters through a port 833 and is distributed by a plenum 841 across a width of the permeate flow channel 843. The retentate leaves through port 832. Recirculating permeate flows in through port 834 and out through port 835. A spacer 840 defines the spacing of structural members 837 from a wall 836 of the permeate flow channel that receives permeate flowing through a filter 838 from a retentate channel 839. The space defines flow bottlenecks such as indicated at 845 where most of the pressure loss occurs in the permeate channel. The arrangement of the structural member 837 may be any suitable arrangement including square lattice structure or hexagonal or circular (in plan view). The assembly shown can be clamped together using fasteners such as bolts passing through openings 831. Appropriate seals may be formed by the compression of clamping.

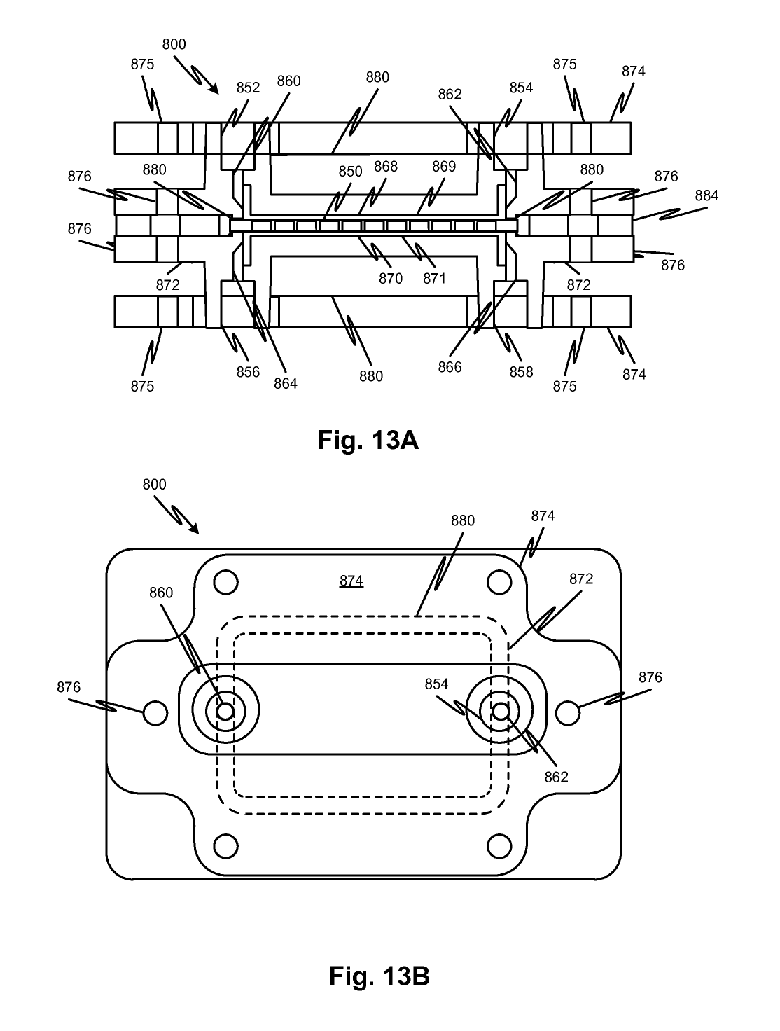

FIGS. 13A and 13B show a separation module 800 has an inlet port 852 for receiving a primary flow to be filtered and an outlet port 854 for a retentate flow to leave. The primary flow channel 869 lies between a wall 868 and a filter 850. A recirculating permeate flow enters through a port 856 and leaves through port 858. The permeate passes through a permeate channel 871 defined between structural members of the filter 850 and a wall 870. The filter is clamped and sealed by continuous ridges 880, which are shown in plan view by dashed lines. The ridge is centered on the axis of the port 852. Flow distribution plenums 860 and 864 spread incoming flows across a width of the channel and gathering plenums 862 and 866 collect across the widths of the channels to convey to the respective ports 854 and 858. A spacer 884 defines the spacing of the channels. The assembly is clamped using fasteners such as bolts passing through holes 875, 876 to pressure plates 874 applying pressure to the ridge 880 which forms a seal around the filter 850. In the present arrangement, it will be observed that the flow of permeate and primary fluid enter and leave directly adjacent the seal such that there are no stagnant regions for the flow. That is the flow channel runs from end to end so there are no dead ends that could cause accumulation of particulates or thrombogenesis in embodiments where the primary fluid is blood or blood products.

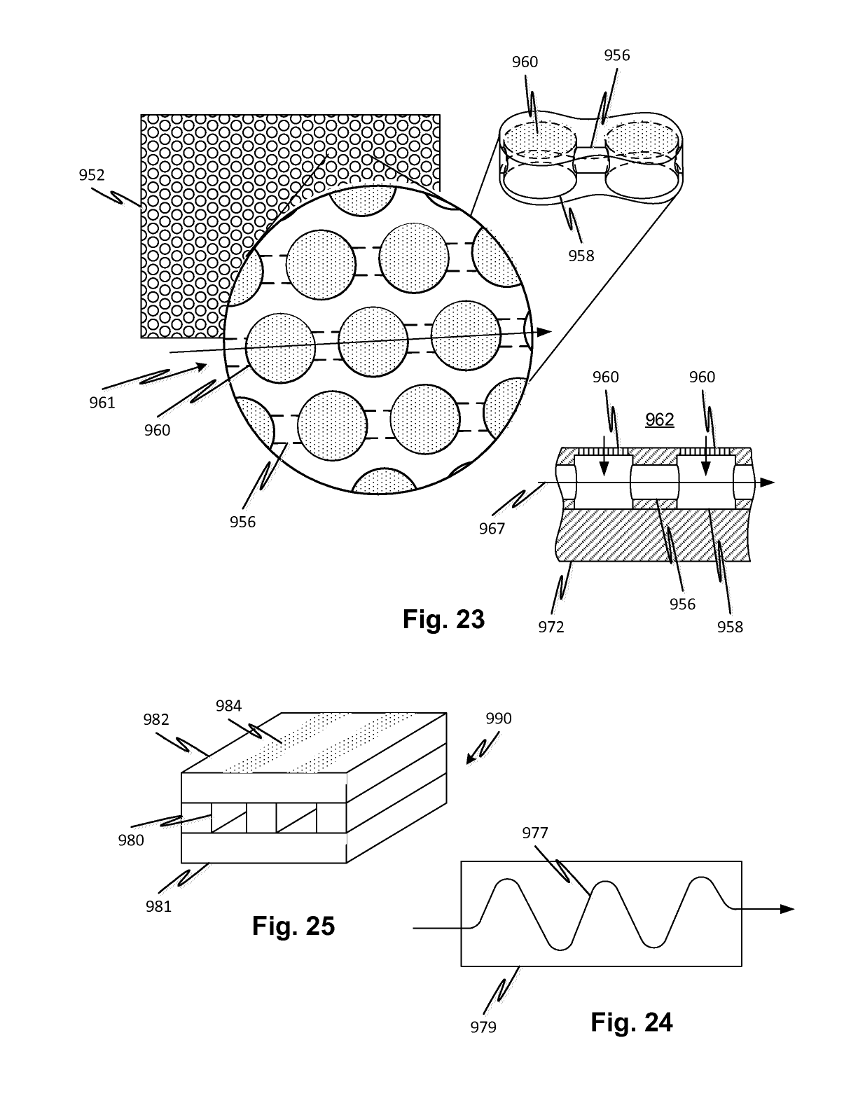

FIG. 23 illustrates a filter configuration in which the permeate flow passes from a retentate flow 962 through channels formed in the filter itself to permeate flow 967. In embodiments, a filter 952 may be fabricated to form channels 956 in which the permeate may flow. In an example embodiment, the structural features described with reference to FIG. 12A and 12B are formed in a hexagonal configuration as illustrated with the filter 960 being formed either integrally or as an attachment. Suitable fabrication techniques are described in U.S. Pat. No. 5,753,014 and US Patent Publication 20080248182, both to van Rijn. A filter with open recesses 958, which form part of the continuous flow path that includes the linking channels 956 and the recesses 958, may be open at the bottom and positioned adjacent a wall 972 of the separation device to close the continuous channel 961. Alternatively, 972 may be formed as a layer of the filter structure itself. Such a filter structure may be readily substituted in the disclosed separation devices. In variations, the channels may be configured such that the flow path meanders through the filter 979 as indicated at 977 in the general concurrent flow direction as illustrated in FIG. 24. Thus, the flow of permeate may not be concurrent with the retentate flow as long as the pressure drop in the streamwise direction of the retentate flow falls stepwise or progressively such that a substantially constant, or at least positive, TMP is maintained for effective use of the filter area. FIG. 25 illustrates a variation of the configuration of FIG. 23 in which elongate channels 982 are formed between a filter layer 982 with pores 984 and bottom layer 981. The bottom layer 981 may be a wall of the separation assembly or an attached or integral structure of the filter 990 itself. The structure may define flow passages 980 such that a gap between the filter 990 and a wall of a permeate channel is not needed. The foregoing filter embodiments may be substituted in any of the method or apparatus embodiments disclosed or claimed.

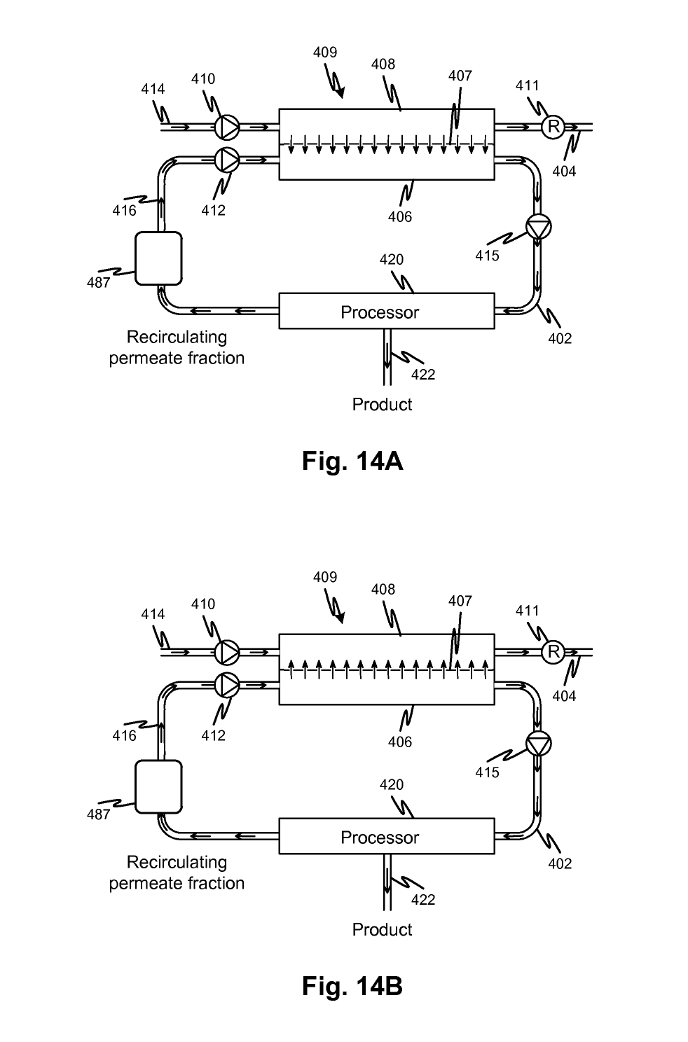

FIG. 14A shows a fluid circuit in which a primary channel receives a flow to be filtered pumped by a pump 410 which is pumped into a retentate channel 408 of a separation module 409. A fraction of the primary flow passes through a filter 407 creating a permeate flow that joins a recirculating permeate flow driven by pump 412 through channel 416. The recirculating permeate flow is pumped through channel 402 through a processor which can cause concentration or imbalance in the composition of the recirculating flow such as concentration of HMW components of plasma as a result of removal of a low molecular weight fraction in a product stream 422. An accumulator 487 stores a fraction of the recirculating permeate for intermittent return through the filter 407 to the retentate channel 408. This flow reversal may be achieved by suitable control of the pumps 412 and 415 or other suitable mechanism to create a positive pressure in the permeate channel 406 relative to the retentate channel 408. The reversed flow through the filter 407 is shown in FIG. 14B. Flow restrictors as indicated at 411 may be used at any point in the circuit as required to provide a pressure balance. The reversal may be performed on a regular schedule based on commands from a controller based on the flow rates and the size of the accumulator 487.

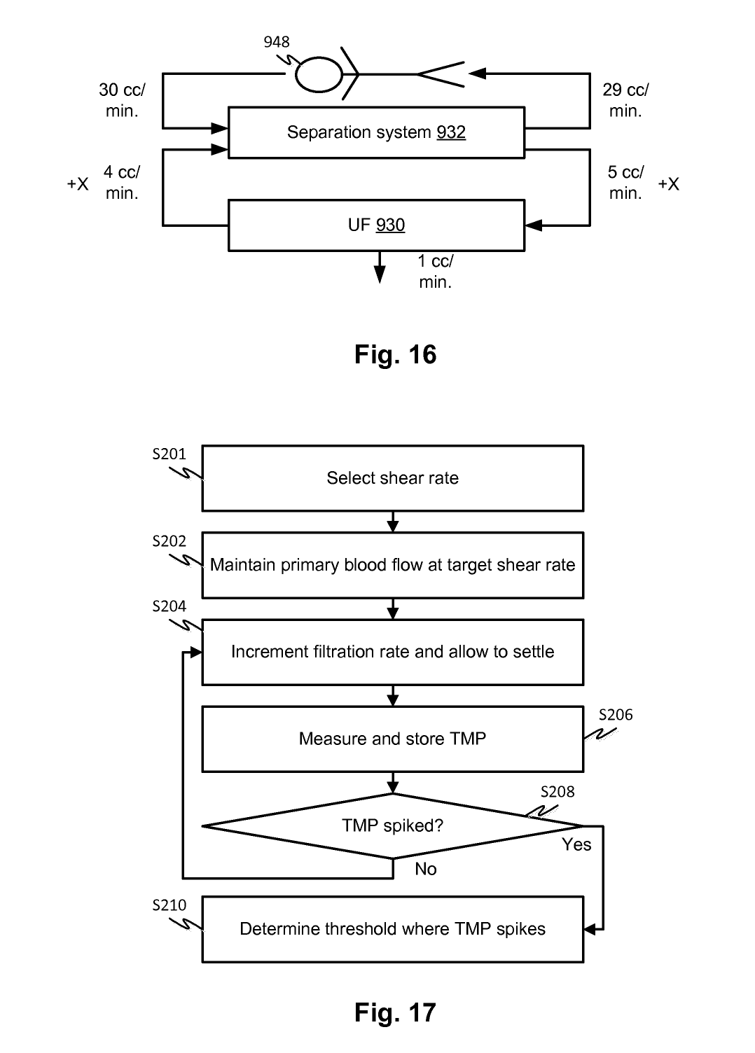

FIG. 16 is a schematic of a blood treatment system for a patient 948 showing example rates for use in an ultrafiltration process in which water and uremic toxins are removed from the patient's blood. These rates are examples only and different treatment process can employ different rates. Blood flows through a separation system 932, which may include separation modules and plasma return components as described with reference to any of the disclosed embodiments. Plasma is ultrafiltered by an ultrafilter 930 to produce a waste flow. The present embodiment is applicable for long term ultrafiltration as may be suited for a portable ultrafiltration process. Blood may be withdrawn from a patient 948 at a rate of 30 cc/min. Plasma is returned to the patient 948 at a rate of 29 cc/min as a result of the blood plasma separation device having an effective volume removal rate of 1 cc/min. The rate of concentrated plasma (reduced water from the ultrafiltration) returned to the patient is 3 cc/min due to the net ultrafiltration rate of 1 cc/min. The added X is the recirculating volume portion which may have, within reason, an arbitrary value. The values shown are illustrative and achievable with a blood plasma separation device having a total wall filter area of about 2 cm².

According to embodiments, the device of FIG. 16 is an ultrafiltration system having a throughput in the range of 0.5 to 3.0 (cc/min)/cm² of filter area. To achieve these throughput rates, the shear rate provided for blood in the blood plasma separation device 932 channel at the surface of the wall filters (now shown here) is in the range of 3,000 to 10,000 sec-1. As discussed below, the rate of withdrawal of plasma depends on the shear rate and the uniformity of the TMP. Within the blood plasma separation device, the filters may have an open area in the range of 3 to 20 percent, a pore size as discussed above with respect to other embodiments, for example, 0.2 to 2 microns, but preferably in the range of 0.5 to 1.5 microns. The blood plasma separation device 932 embodiment of FIG. 16 may have multiple layers but may be a single layer channel with a single filter.

In a preferred configuration, the maximum filtration rate is empirically established for the particular embodiment (a method of establishing the maximum filtration rate is described below) including the particular wall filters, blood channel height, and other parameters for a single channel device in order to omit the influence of instabilities or other dependencies on the plumbing of the blood plasma separation device as a whole. The maximum filtration rate may be determined according to a selected shear rate which is established based on blood properties and other medical factors, for example, such as safety and tolerance for hemolysis. For example, anemic patients may have a low tolerance for hemolysis by the blood treatment. In an example, a shear rate of 7500 sec-1 may be used and the single layer blood plasma separation device operated to determine the maximum filtration rate, for example using the procedure of FIG. 17. In the preferred treatment configuration, the device of FIG. 16 is operated at a filtration rate that is substantially below this maximum rate and in a preferred embodiment, at a rate of about 50% of the maximum filtration rate of plasma. This reduced rate has been experimentally determined to allow for the reliable operation of multilayer devices whose actual performance in a multilayer blood plasma separation device has been determined to be lower than predicted by multiplying the single layer throughput by the number of layers. Operating above the 50% reduced rate has been found experimentally to produce malfunctions in multilayer devices and a reduced rate (relative to the experimentally determined maximum) has proved exhibit reliable function.

Although the embodiment of FIG. 16 and elsewhere herein are used for blood plasma separation, it should be clear, as stated elsewhere in the present disclosure, that the blood plasma separation device may be used in other devices and systems for the extraction of plasma or for the filtration of fluids from other kinds of suspensions. For blood processing, for example, the blood plasma separation device may be employed in dialysis, hemodiafiltration, continuous renal replacement therapy, apheresis, sepsis mitigation, etc. In such other systems, the plasma may be separated and subjected to some secondary processing before being returned to the body and may not involve the removal of bulk fluid such as water and small molecules. For example, a secondary processor including a filter cascade, chemical treatment, adsorption treatment, etc. may be substituted for the ultrafilter 930. The blood plasma separation device may also be used for generating samples of plasma for real time testing as described above or for plasma pheresis. The blood plasma separation device of FIG. 23 may be substituted for any of the foregoing blood plasma separation device devices in any system described elsewhere in the present application.

FIG. 17 shows a procedure for determining a maximum filtrate flow rate according to embodiments of the disclosed subject matter. A shear rate is selected S201 responsively to the maximum tolerable, for example the rate at which hemolysis may occur. As mentioned above, a target shear rate of blood flow is maintained in a single layer blood plasma separation device as shown at S202. An initial plasma filtration rate is established as indicated at S204 and in later stages of the process, incremented by a small amount. After a period of time which allows the filtration process to settle, during which TMP may be measured, a TMP measurement is stored S206. In this process, it has been found experimentally that at some filtration rate, the TMP will start to rise dramatically just prior to the achievement of the maximum filtration rate. If this condition arises, it will be immediately apparent and serves as a terminating condition for the process as indicated at S208 and the maximum TMP can be determined as the rate just prior to which the TMP spiked. The value may be derived by interpolation as well. Prior to the spike termination condition, steps S204, S206, and S208 are repeated.

Note that in the embodiment of FIG. 16 and all other embodiments in which the separation module is described as being used with a processing device such as an ultrafilter, other treatment devices are also possible. In FIG. 22, for example, a dialyzer 936 is shown is used to exchange solutes with, and remove or add water to/from the recirculating plasma. The dialyzer 936 may be operated in diafiltration mode as well. In further variations, the replacement fluid 937 is added to the recirculating plasma or infused directly the patient in a treatment mode based on hemofiltration. Other blood treatment variations may be evident to those of skill in the art based on existing and future extracorporeal blood treatment modes by replacing direct treatment of blood with treatment of plasma in the circulating loop. Examples include plasmapheresis, hemodiafiltration, etc.

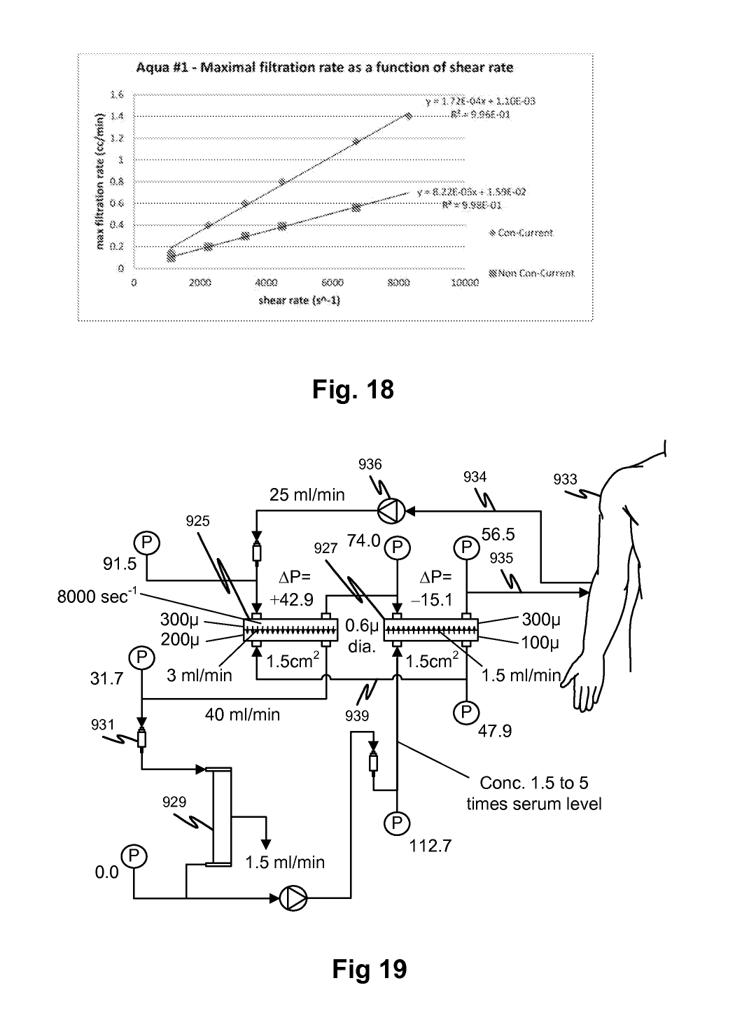

FIG. 18 shows a result of measurements generated according to the process of FIG. 17. The chart shows TMP over a period of time during which the filtration rate was ramped up in steps until the termination condition was in evidence. These data show an example run only and is not representative of the highest possible TMP that can be achieved with a particular filter under different conditions, for example, a higher shear rate or more uniform control of TMP.

FIG. 19 shows example quantitative data for an example ultrafiltration embodiment superimposed on a schematic of the fluid circuit. The fluid circuit employs two filter modules, one 925 for primary blood/plasma separation and another 927 for the return of concentrated plasma to a return stream. Blood is drawn by a pump 936 through an arterial line 934 and flows through a pressure pulse damper 931 (only one damper is labeled to keep the drawing from being cluttered). The blood flows into the separation module 925 where plasma is removed and the result flows into the filter module 927 where concentrated plasma is forced into it before returning the blood via the venous line 935. A recirculating loop 939 returns a major fraction of the flow from the filter module 927 to a recirculating stream supplied to separation module 925 which functions to provide a downstream pressure profile to maintain constant TMP and pick up additional plasma which is fed through a damper 931 and to an ultrafilter 929 where a product stream (waste) is removed. The recirculating flow then enters the filter module 927 to close the loop. Pressures at points in the loops are shown by the pressure gauge symbols (circle-P) in units Torr. The average TMP in the separation module 925 is 42.9. The return TMP for concentrated plasma return in the filter module 927 is 15.1. The pore size of the filters used in both modules 925 and 927 is 0.6 micron. The depth of the retentate channel of separation module 925 is 300 microns and that of the permeate channel 200 microns. The filter module 927 upstream (filtrand) side has a depth of 100 microns and the downstream (filtrate) side a depth of 300 microns. The blood flow rate is 25 ml/min and the recirculating plasma rate is 40 ml/min. The waste flow rate is 1.5 ml/min. The rate of flow of permeate through the filter of separation module 925 is 3 ml/min and the return rate of concentrated plasma through the filter of the filter module 927 is 1.5 ml/min. The areas of the filters of both modules 925 and 927 is 1.5 cm². The recirculating plasma may be concentrated in HMW blood components, for example serum albumin, to a level that is 1.5 to 5 times the serum levels. It has been confirmed experimentally that effective ultrafiltration is possible with concentrations that are in the range 2 to 3 times the serum level.

In the disclosed embodiments, the processor may be replaced by an adsorption device, a further filter, an ultrafilter (e.g., dialyzer), diafilter, or other processing device. In any of the embodiments, the pumps may be peristaltic pumps. In any of the embodiments, flow dampeners may be used to mitigate pressure pulses due to the pumps.

It has been observed that in filtering whole blood, a layer of cytoplasmic bodies, principally erythrocytes, accumulates on the cross-flow filter. This may result in a passivation of the surface of the filter. It does add to the flow resistance of the filter and observations demonstrate that the porosity of the filter is not a significant design parameter within the range of porosities tested.

In example embodiments, the performance of the filtration device has been confirmed with whole blood. A cross-flow filter having 1.56 cm² active area, with pores of a slot configuration, 0.6 μ wide by 2 μ long was employed in tests. Blood and plasma were pumped in parallel flows on opposite sides of the cross-flow filter to effect a continuous cross-flow achieving up to 1.5 ml/min. The following table shows examples of tests over periods of 1.5 hrs. comparing maximum flow of plasma across the cross-flow filter using concurrently flowing permeate (plasma) and flow without the concurrent flow.

In any of the foregoing embodiments, the crossflow filter may be fabricated from any suitable material. For example, organic and inorganic materials and composites including polymers, materials, ceramic, metals, etc. Examples of materials are listed in U.S. Pat. No. 5,753,014.

Note that in any of the embodiments, patient accesses may take the form of any type of access including a fistula, central line, and employ one or more needles, cannulae, or catheters, mulitiple-lumen cannulae or catheters or other devices. The illustration showing an arm should be understood as symbolic of any type of blood access device.

According to embodiments, the disclosed subject matter includes a filtration apparatus with a crossflow filter that has a retentate channel with inlet and outlet ends and a permeate channel adjacent to the retentate channel with inlet and outlet ends. The permeate and retentate channels are separated by a crossflow filter. A recirculation channel connects the permeate channel outlet end with the permeate channel inlet end. The recirculation channel is connected to the permeate channel outlet end through a treatment component that alters a property of the flow in the recirculation channel. A property control device extracts a fraction of the flow in the recirculation channel at a flow rate that maintains constant property of the flow in the recirculation channel. At least one pump may be used in the recirculation channel for flowing fluid therethrough.

The property control device may include a check valve or it may include in addition or alternatively, a filter that separates the recirculation channel from a flow emanating from the retentate channel outlet. The property control device may also include a cross-flow filter having similar construction to that of the crossflow filter used for primary separation. In embodiments, the property control device may be configured to sustain a constant level of a property in the recirculation channel by continuously removing a fraction of the fluid therein, which is replaced by fresh permeate, thereby creating a balance. For example, if the property modified by the treatment component is an ultrafilter filtrate fractions remaining in the recirculation channel may concentrate to a higher level than in the permeate flowing into the recirculation channel. By drawing off a fraction of the flow in the recirculation channel, the continuous replenishment with permeate from the crossflow filter allows a balanced composition to be maintained, e.g., in this case, a predefined level of concentration of the ultrafilter's filtrate. In further variations of the above embodiment, a pump may be connected to the retentate channel inlet. A blood access line may be connected to the retentate channel inlet. The permeate channel may have a tapered cross-section. The retentate and permeate channels may have tapered cross-sections such that the retentate channel diminishes in cross-sectional area and the permeate channel increases in cross-sectional area. The cross-flow filter, pumps and channels may be sized such that a stable flow of blood plasma through the cross-flow filter may be achieved with a flow of blood in the retentate channel. The permeate channel of the crossflow filter may have a series of spaced structural members that restrict flow at a point coinciding therewith and which receive permeate from the retentate channel between them. The structural members may be configured to stiffen the filter. The filter may have a smooth flat polished surface that helps to keep retentate particles from adhering to the filter surface. The filter may have regularly spaced straight pores with an aspect ratio (axial length of pore to diameter) of no more than ten. The aspect ratio may be less than 5 or may be no more than two. The pores may be straight, non-branching channels. The property control device may include a fluid connector configured to direct a fraction of a flow in the recirculation channel directly to a patient. The recirculating flow can be returned to the patient by a variety of means for example a dual lumen catheter in a patient central line may be used to flow retentate and a fraction of the recirculating permeate directly to the patient's access. Alternatively, the two flows can be combined and flowed through a single lumen catheter into the patient. For example, the two flows may converge in a junction which is attached at the base of the junction to the single lumen. A triple lumen catheter may be used to draw the primary flow from the patient and return the recirculating permeate and crossflow filtered retentate back to respective lumens of the triple lumen catheter or cannula.

According to embodiments, the disclosed subject matter includes a method for cross-flow filtering plasma from whole blood while maintaining a positive transmembrane pressure of a streamwise length of a filter used for the cross-flow filtering. The method includes removing ultrafiltrate from the plasma resulting from the cross-flow filtering and returning a result of the removing to a source of the whole blood. The filter may have non-branching channels. The filter may have straight channels therethrough with uniform pore size between 0.02 micron and 2 microns. The maintenance of positive pressure may include co-flowing plasma on a permeate side of the filter such that a pressure drop in a streamwise direction is maintained. The streamwise flow on the permeate side of the filter produces a pressure drop along the filter that compensates for a pressure drop along the retentate side. The method may include returning ultrafiltrate to a permeate side of the filter to generate a flow generating a streamwise pressure drop therealong. The plasma flow through the cross-flow filter may be in the range of 0.1 to 10 ml/min. The flow rate of plasma through the cross-flow filter may be between 5 and 25 percent of the rate of flow of whole blood into the filtration device. The cross-flow filtering may include flowing blood in a microfluidic channel having a depth less than 500 microns. In embodiments, the channel may have a depth less than 300 microns. The cross-flow filtering may include compacting a layer of blood cells on the surface of a cross-flow filter. The maintenance of positive pressure may aided by co-flowing plasma on a permeate side of the filter such that a pressure drop in a streamwise direction maintained. The method may further include concentrating blood proteins in the co-flowing plasma and returning a result thereof to the source of blood such that the net flow of cross-flow filtered plasma is equal to the net flow of ultrafiltrate and a net flow of the returning. The returning may include flowing the plasma resulting from the concentrating through a filter back to a patient blood stream. The maintaining may be effective to provide a constant transmembrane pressure over an entirety of a cross-flow filter. The cross-flow filtering may include flowing whole blood through a channel having a depth of less than 500 microns. The cross-flow filtering may include flowing whole blood through a channel having a depth of 200 microns or less. The cross-flow filtering may include flowing whole blood through a retentate channel having a depth of less than 500 microns, wherein the maintaining includes flowing recirculated plasma through a channel whose depth is less than the retentate channel depth. The cross-flow filtering may include flowing whole blood through a retentate channel having a depth of less than 500 microns, wherein the maintaining includes flowing recirculated plasma through a channel whose depth is about half the retentate channel depth. A flow of plasma across a filter in the cross-flow filtering may be greater than 0.5 cm³/cm² of cross-flow filter area.

According to embodiments, the disclosed subject matter includes a method of cross-flow filtering a suspension having a particle volume fraction of at least 1 percent. The method includes cross-flow filtering the suspension by flowing permeate from a retentate side of a filter to a permeate side while flowing the suspension along the retentate side. The method further includes generating a pressure drop along the permeate side of the filter by recirculating a portion of the permeate across the filer permeate side. In this embodiment, the generating is effective to create a positive transmembrane pressure over an entirety of the cross-flow filter used to filter the suspension. The method may include extracting a product stream from a flow on the permeate side.

According to embodiments, the disclosed subject matter includes a microfluidic separation device with a fluid circuit device having multiple flow channels fed from a common fluid header. Each flow channel has parallel facing opposing walls separated by a separation distance of 500 microns or less. Each flow channel has an inlet and a plurality of outlet openings along the walls spanning a streamwise span of the walls. A fluid delivery system is connected to the flow channel and configured to deliver a predefined fluid to the flow channel. The predefined fluid is a fluid with suspended particles which exhibits the property of there being a predefined maximum filtration rate through the plurality of outlet openings for a given shear rate across the plurality of openings. A controller is configured to regulate flow rates of the fluid delivery system to control a filtrand flow rate through the at least one flow channel and to control a filtrate flow rate through the outlet openings at at least 10% below the predefined maximum filtration rate. The predefined fluid may be blood and the fluid delivery system may include a patient vascular access. A processor may be configured for receiving the filtrate from the fluid circuit device. An ultrafilter may be configured to receive the filtrate from the fluid circuit device and return concentrated filtrate to the patient. The flow channel may be a rectangular flow channel, and the walls may be facing opposing walls whose widths are at least ten times the separation distance between them.

According to embodiments, the disclosed subject matter includes a microfluidic separation method that includes providing a fluid circuit device with multiple flow channels, each having parallel facing opposing walls separated by a separation distance of 500 microns or less, wherein each flow channel has an inlet and a plurality of outlet openings along the walls spanning a streamwise span of the walls. The method includes delivering a fluid suspension to the flow channel, wherein the fluid suspension is one that exhibits a property of there being a predefined maximum filtration rate through the plurality of outlet openings for a given shear rate across the filter. The method includes regulating a flow rate of the fluid suspension through the flow channel at a rate corresponding to a predefined shear rate and regulating a filtrand flow rate through the plurality of openings at at least 10% below the predefined maximum filtration rate. The predefined fluid may be blood and the fluid delivery system may include a patient vascular access.

The method may further include flowing filtrate through a processor configured for receiving the filtrate from the fluid circuit device. The method may include ultrafiltering filtrate from the plurality of openings and returning concentrated filtrate to the patient. The predefined shear rate may be at least 2000 sec-1. The predefined shear rate may be at least 3000 sec-1, 5000 sec-1, or at least 7000 sec-1 in respective embodiments.

According to embodiments, the disclosed subject matter includes a system for ultrafiltering blood having a crossflow filtration apparatus with a crossflow filter configured to separate plasma from blood received through an arterial blood line. The crossflow filtration apparatus is configured to recirculate plasma in a recirculation channel connecting a plasma permeate outlet of the crossflow filtration apparatus to a plasma permeate recirculating flow connected to an inlet of the cross flow filtration apparatus. A plasma pump in the recirculation channel is configured to maintain a flow therein such as to maintain a substantially constant transmembrane pressure at all points of a surface of the crossflow filter. The channel has an ultrafilter arranged to remove water from a flow from plasma in the channel and a return filter separating the recirculating channel from a venous blood line connected to a retentate outlet of the crossflow filtration apparatus. A blood pump along with the plasma pump are arranged to flow plasma from the recirculating channel through the return filter.

The crossflow filtration apparatus may have a permeate channel arranged for concurrent flow of recirculating plasma with retentate flow therethrough. The permeate channel may have spaced structural members supporting the filter and a flat wall beneath each of them that define flow bottlenecks where most of the pressure drop along the permeate channel occurs.

According to embodiments, the disclosed subject matter includes a method of treating blood that includes determining a maximum shear rate based on a minimum shear rate causing damage to precious components of blood. Thus the flow rate may be selected so as to provide the highest rate that does not damage cells including a safety margin. The maximum shear rate thus lies below the minimum shear rate. The method includes determining a critical transmembrane pressure of a crossflow filter subjected to the maximum shear during crossflow filtration thereof. For blood processing the critical pressure may lie at a point where in spite of the shear, the flexible cells get trapped in pores of the crossflow filter causing a reduction in permeate rate through the filter and with constant volume flow, an accelerating transmembrane pressure that causes the entire filter to clog up. The critical transmembrane pressure causes an abrupt diminution in a relationship between flow across the crossflow filter and the applied transmembrane pressure, indicating a loss of efficiency of the crossflow filter throughput. The crossflow filter is configured to retain at least erythrocytes. The method includes crossflow filtering blood through a crossflow filter at an operating transmembrane pressure determined responsively to the critical transmembrane pressure to remove at least erythrocytes. The method includes ultrafiltering the permeate resulting from the crossflow filtering and returning ultrafiltered permeate and blood to a patient. The method further includes performing the foregoing crossflow filtering, ultrafiltering, and returning continuously for at least a day.

A flux rate of permeate passing through the crossflow filter may be between 0.5 and 2 ml/cm² of filter area. The flow rate of permeate passing through the crossflow filter may be between 0.5 and 5 ml/min. The flow of ultrafiltrate in the ultrafiltering may be at a rate between 0.5 and 5 ml/min. The returning may include passing the permeate through a return filter to a venous return line. The crossflow filtering may include passing blood through a retentate channel with a depth of less than 500 microns. The crossflow filtering may include flowing a recirculating stream of permeate through a channel underlying a permeate side of the crossflow filter to generate a pressure drop through the channel that maintains the transmembrane pressure determined responsively to the critical transmembrane pressure. The rate of flow of permeate through the channel may be greater than a rate of flow of blood across a retentate side of the crossflow filter. The permeate may be substantially plasma. The ultrafiltering may produce a waste stream of water and aqueous solutes.

The crossflow filter may have a polished flat surface on a retentate side thereof. The crossflow filter may have an array of pores of 0.2 to 2.0 micron diameter. The crossflow filter may have pores whose depth may be not more than 5 times their diameters. The crossflow filter may be supported by structural members that restrict a flow of permeate across them to produce a stepwise pressure profile in a permeate channel underlying the crossflow filer. The crossflow filtering may be performed using a single crossflow filter whose area may be not more than 5 cm and a single retentate channel and a single permeate channel. The cross sectional area of a retentate channel overlying the crossflow filter may progressively diminish in a streamwise direction. The cross sectional area of a permeate channel underlying the crossflow filter may progressively expand in a streamwise direction. The width of a retentate channel overlying the crossflow filter may progressively diminish in a streamwise direction. The width of a permeate channel underlying the crossflow filter may progressively expand in a streamwise direction. The returning may include passing the permeate though a check valve. The returning may include passing the permeate through a return filter to a venous blood return line, wherein the crossflow filter and the return filter are arranged in a single module. The operating transmembrane pressure determined responsively to the critical transmembrane pressure may be determined responsively to a minimum shear rate required to sweep erythrocytes from a retentate side of the crossflow filter at a given transmembrane pressure.

The crossflow filtering may be effective to sweep erythrocytes from a retentate side of the crossflow filter at the operating transmembrane pressure. The crossflow filtering may include flowing retentate and permeate concurrently on both sides of the crossflow filter. The retentate may flow across the crossflow filter in a rectangular channel having an aspect ratio of at least ten.

According to embodiments, the disclosed subject matter includes a method of treating blood that includes determining a maximum shear rate based on a minimum shear rate causing damage to precious components of blood, where the maximum shear rate lies below the minimum shear rate. The method includes determining a critical transmembrane pressure of a crossflow filter subjected to the maximum shear during crossflow filtration thereof. The critical transmembrane pressure is one which causes an abrupt diminution in a relationship between flow across the crossflow filter and the applied transmembrane pressure, indicating a loss of efficiency of the crossflow filter throughput. The crossflow filter is configured to retain at least erythrocytes. The crossflow filtering blood through a crossflow filter is controlled to be at an operating transmembrane pressure determined responsively to the critical transmembrane pressure to remove at least erythrocytes therefrom. The method includes processing the permeate resulting from the crossflow filtering and returning processed permeate and blood to a patient. The method further includes performing the foregoing crossflow filtering, processing, and returning continuously for at least a day.

According to further embodiments, the processing may include adsorbing, ultrafiltering, dialyzing, hemofiltering, or hemodiafiltering the permeate. The flux rate of permeate passing through the crossflow filter may be between 0.5 and 2 ml/cm² of filter area. The flow rate of permeate passing through the crossflow filter may be between 0.5 and 5 ml/min.

The returning may include passing the permeate through a return filter to a venous return line. The crossflow filtering may include passing blood through a retentate channel with a depth of less than 500 microns. The crossflow filtering may include flowing a recirculating stream of permeate through a channel underlying a permeate side of the crossflow filter to generate a pressure drop through the channel that maintains the transmembrane pressure determined responsively to the critical transmembrane pressure. The rate of flow of permeate through the channel may be greater than a rate of flow of blood across a retentate side of the crossflow filter. The permeate may be substantially plasma. The ultrafiltering may produce a waste stream of water and aqueous solutes.

The crossflow filter may have a polished flat surface on a retentate side thereof. The crossflow filter may have an array of pores of 0.2 to 2.0 micron diameter. The crossflow filter may have pores whose depth may be not more than 5 times their diameters. The crossflow filter may be supported by structural members that restrict a flow of permeate across them to produce a stepwise pressure profile in a permeate channel underlying the crossflow filer. The crossflow filtering may be performed using a single crossflow filter whose area may be not more than 5 cm and a single retentate channel and a single permeate channel. The cross sectional area of a retentate channel overlying the crossflow filter may progressively diminish in a streamwise direction. The cross sectional area of a permeate channel underlying the crossflow filter may progressively expand in a streamwise direction. The width of a retentate channel overlying the crossflow filter may progressively diminish in a streamwise direction. The width of a permeate channel underlying the crossflow filter may progressively expand in a streamwise direction. The returning may include passing the permeate though a check valve. The returning may include passing the permeate through a return filter to a venous blood return line, wherein the crossflow filter and the return filter are arranged in a single module. The operating transmembrane pressure determined responsively to the critical transmembrane pressure may be determined responsively to a minimum shear rate required to sweep erythrocytes from a retentate side of the crossflow filter at a given transmembrane pressure.

The crossflow filtering may be effective to sweep erythrocytes from a retentate side of the crossflow filter at the operating transmembrane pressure. The crossflow filtering may include flowing retentate and permeate concurrently on both sides of the crossflow filter. The retentate may flow across the crossflow filter in a rectangular channel having an aspect ratio of at least ten.

According to embodiments, the disclosed subject matter include a method for extracorporeal treatment of blood, comprising: flowing whole blood at a primary flow rate from a patient in a crossflow filter and extracting as permeate, a plasma flow with a volume fraction of the whole blood flow of 1 to 25 percent and returning a reduced flow of blood, resulting from the extracting, back to the patient. The method includes recirculating the plasma flow to the crossflow filter at a rate effective to moderate a change in transmembrane pressure across the crossflow filter and controlling a tonicity of the recirculating plasma flow to a level above that of the whole blood. The controlling includes continuously returning hypertonic plasma to the patient at a predefined extraction rate removing water and uremic toxins from the recirculating plasma at a predetermined ultrafiltration rate.

The predefined extraction rate may be between 10 and 75 percent of a rate of flow of permeate. The predefined extraction rate may be between 30 and 70 percent of a rate of flow of permeate. The predefined extraction rate may be between 40 and 60 percent of a rate of flow of permeate. The predefined ultrafiltration rate may be between 10 and 75 percent of a rate of flow of permeate. The predefined ultrafiltration rate may be between 30 and 70 percent of a rate of flow of permeate. The predefined ultrafiltration rate may be between 40 and 60 percent of a rate of flow of permeate. The rate of permeate flow may be between 5 and 25 percent of the primary rate. The rate of permeate flow may be between 10 and 20 percent of the primary rate. The crossflow filter may have a pore size between 400 and 800 nm. The flowing whole blood may be effective to immobilize red blood cells on a retentate side of the crossflow filter. The crossflow filter may have a regular array of unlinked, non-branching, pores each of which may have an aspect ratio of length to diameter of less than 5. The crossflow filter may have a regular array of unlinked, non-branching, pores each of which may have an aspect ratio of length to diameter of less than 2. The tonicity of the recirculating plasma flow may be between 1.5 and 5 times that of the whole blood.

According to embodiments, the disclosed subject matter include a method for treating blood of a patient including extracting plasma from whole blood from the patient to form a flow of freshly extracted plasma. The method includes ultrafiltering the freshly extracted plasma to produce a flow of dewatered plasma. The method includes directly combining the freshly extracted plasma with the dewatered plasma and flowing the combined freshly extracted and dewatered plasma back to the patient. The flowing the combined freshly extracted plasma may include further dewatering the combined freshly extracted and dewatered plasma back and then flowing a fraction thereof back to the patient while retaining a fraction as dewatered plasma to be combined with freshly extracted plasma.

The directly combining may include generating a flow in a crossflow filter channel that moderates a streamwise change in transmembrane pressure along a retentate side of a crossflow filter used in the extracting plasma. The extracting may include flowing the blood in a microfluidic channel whose height is no more than 500 microns in depth. The microfluidic channel may be on a retentate side of a crossflow filter and the extracting may be at a rate of 1 to 5 ml/min. The microfluidic channel may be on a retentate side of a crossflow filter and the extracting may be at a rate of at least 1 ml/min-cm² of filter area.

In any of the foregoing method, system, or apparatus embodiments, the cross flow filter may be further limited to filters whose pore spacing is such that a layer of immobilized cells may be separated by such a distance that the immobilized cells can protect the patency of pores that are not blocked. If the spacing is too wide, then no adjacent cells can keep other cells from blocking neighboring cells. Thus, in treating blood according to the disclosed embodiments, a pore spacing that permits cells trapped in pores to protect other pores from being blocked may be desirable in embodiments.

It is, thus, apparent that there is provided, in accordance with the present disclosure, methods, devices, and systems for fluid separation. Many alternatives, modifications, and variations are enabled by the present disclosure. Features of the disclosed embodiments can be combined, rearranged, omitted, etc., within the scope of the invention to produce additional embodiments. Furthermore, certain features may sometimes be used to advantage without a corresponding use of other features. Accordingly, Applicants intend to embrace all such alternatives, modifications, equivalents, and variations that are within the spirit and scope of the present invention.

1-80. (canceled)

81. A method of treating blood, comprising:
determining a maximum shear rate based on a minimum shear rate causing damage to precious components of blood, the maximum shear rate lying below said minimum shear rate;
determining a critical transmembrane pressure of a crossflow filter subjected to said maximum shear during crossflow filtration thereof, said critical transmembrane pressure being one which causes an abrupt diminution in a relationship between flow across the crossflow filter and the applied transmembrane pressure, indicating a loss of efficiency of the crossflow filter throughput;
the crossflow filter being configured to retain at least erythrocytes;
crossflow filtering blood through a crossflow filter at an operating transmembrane pressure determined responsively to said critical transmembrane pressure to remove at least erythrocytes therefrom;
processing the permeate resulting from said crossflow filtering;
returning processed permeate and blood to a patient;
performing said foregoing crossflow filtering, processing, and returning continuously for at least a day.

82. The method of claim 81, wherein the processing includes adsorbing, ultrafiltering, dialyzing, hemofiltering, or hemodiafiltering said permeate.

83. The method of claim 81, wherein flux rate of permeate passing through the crossflow filter is between 0.5 and 2 ml/cm² of filter area.

84. The method of claim 81, wherein the flow rate of permeate passing through the crossflow filter is between 0.5 and 5 ml/min.

85. The method of claim 81, wherein said returning includes passing the permeate through a return filter to a venous return line.

86. The method of claim 81, wherein the crossflow filtering includes passing blood through a retentate channel with a depth of less than 500 microns.

87. The method of claim 81, wherein the crossflow filtering includes flowing a recirculating stream of permeate through a channel underlying a permeate side of said crossflow filter to generate a pressure drop through said channel that maintains said transmembrane pressure determined responsively to said critical transmembrane pressure.

88. The method of claim 87, wherein the rate of flow of permeate through said channel is greater than a rate of flow of blood across a retentate side of said crossflow filter.

89. The method of claim 81, wherein said permeate is substantially plasma.

90. The method of claim 81, wherein said crossflow filter has a polished flat surface on a retentate side thereof.

91. The method of claim 81, wherein said crossflow filter has an array of pores of 0.2 to 2.0 micron diameter.

92. The method of claim 81, wherein said crossflow filter has pores whose depth is not more than 5 times their diameters.

93. The method of claim 81, wherein the crossflow filter is supported by structural members that restrict a flow of permeate across them to produce a stepwise pressure profile in a permeate channel underlying said crossflow filer.

94. The method of claim 81, wherein crossflow filtering is performed using a single crossflow filter whose area is not more than 5 cm and a single retentate channel and a single permeate channel.

95. The method of claim 81, wherein cross sectional area of a retentate channel overlying said crossflow filter progressively diminishes in a streamwise direction.

96. The method of claim 81, wherein cross sectional area of a permeate channel underlying said crossflow filter progressively expands in a streamwise direction.

97. The method of claim 81, wherein width of a retentate channel overlying said crossflow filter progressively diminishes in a streamwise direction.

98. The method of claim 81, wherein width of a permeate channel underlying said crossflow filter progressively expands in a streamwise direction.

99. The method of claim 81, wherein the returning includes passing said permeate though a check valve.

100. The method of claim 81, wherein said returning includes passing the permeate through a return filter to a venous blood return line, wherein the crossflow filter and the return filter are arranged in a single module.

101. The method of claim 81, wherein said operating transmembrane pressure determined responsively to said critical transmembrane pressure is determined responsively to a minimum shear rate required to sweep erythrocytes from a retentate side of the crossflow filter at a given transmembrane pressure.

102. The method of claim 81, wherein crossflow filtering is effective to sweep erythrocytes from a retentate side of the crossflow filter at the operating transmembrane pressure.

103. The method of claim 81, wherein the crossflow filtering includes flowing retentate and permeate concurrently on both sides of the crossflow filter.

104. The method of claim 81, wherein retentate flows across said crossflow filter in a rectangular channel having an aspect ratio of at least ten.

105. A method for extracorporeal treatment of blood, comprising:
flowing whole blood at a primary flow rate from a patient in a crossflow filter and extracting as permeate, a plasma flow with a volume fraction of the whole blood flow of 1 to 25 percent and returning a reduced flow of blood, resulting from said extracting, back to the patient;
recirculating the plasma flow to the crossflow filter at a rate effective to moderate a change in transmembrane pressure across said crossflow filter;
controlling a tonicity of the recirculating plasma flow to a level above that of the whole blood;
the controlling including continuously returning hypertonic plasma to the patient at a predefined extraction rate removing water and uremic toxins from the recirculating plasma at a predetermined ultrafiltration rate.

106. The method of claim 105, wherein the predefined extraction rate is between 10 and 75 percent of a rate of flow of permeate.

107. The method of claim 105, wherein the predefined extraction rate is between 30 and 70 percent of a rate of flow of permeate.

108. The method of claim 105, wherein the predefined extraction rate is between 40 and 60 percent of a rate of flow of permeate.

109. The method of claim 105, wherein the predefined ultrafiltration rate is between 10 and 75 percent of a rate of flow of permeate.

110. The method of claim 105, wherein the predefined ultrafiltration rate is between 30 and 70 percent of a rate of flow of permeate.

111. The method of claim 105, wherein the predefined ultrafiltration rate is between 40 and 60 percent of a rate of flow of permeate.

112. The method of claim 105, wherein the rate of permeate flow is between 5 and 25 percent of said primary rate.

113. The method of claim 105, wherein the rate of permeate flow is between 10 and 20 percent of said primary rate.

114. The method of claim 105, wherein the crossflow filter has a pore size between 400 and 800 nm.

115. The method of claim 105, wherein the flowing whole blood is effective to immobilize red blood cells on a retentate side of said crossflow filter.

116. The method of claim 105, wherein the crossflow filter has a regular array of unlinked, non-branching, pores each of which has an aspect ratio of length to diameter of less than 5.

117. The method of claim 105, wherein the crossflow filter has a regular array of unlinked, non-branching, pores each of which has an aspect ratio of length to diameter of less than 2.

118. The method of claim 105, wherein the tonicity of the recirculating plasma flow is between 1.5 and 5 times that of the whole blood.

119-127. (canceled)
